(19) United States
(12) Patent Application Publication
(10) Pub. No.: US 2025/0044305 A1
(43) Pub. Date: Feb. 6, 2025

(54) LIQUID CHROMATOGRAPHY TANDEM-MASS SPECTROMETRY (LC-MS/MS) ANALYSIS METHOD FOR DETECTING 11 VITAMINS D IN BLOOD

(71) Applicant: Beijing Harmony Health Medical Diagnostics Co., Ltd., Beijing (CN)

(72) Inventors: Yongjuan JIA, Beijing (CN); Chunran LIU, Beijing (CN); Xingli LIU, Beijing (CN); Jie ZHANG, Beijing (CN); Junjun NI, Beijing (CN)

(73) Assignee: Beijing Harmony Health Medical Diagnostics Co., Ltd., Beijing (CN)

(21) Appl. No.: 18/382,160

(22) Filed: Oct. 20, 2023

(30) Foreign Application Priority Data
Aug. 4, 2023 (CN) 202310982084.2

(51) Int. Cl. G01N 33/82; G01N 30/72

(52) U.S. Cl. CPC G01N 33/82; G01N 30/7233

(57) ABSTRACT

An LC-MS/MS method for detecting 11 vitamins D in blood is provided. The 11 vitamins D in the invention include D2, D3, 1,25-(OH)₂D2, 1,25-(OH)₂D3, 25-(OH)D2, 25-(OH)D3, 3-epi-25-(OH)D2, 3-epi-25(OH)D3, 24,25-(OH)₂D2, 24,25-(OH)₂D3 and 3-epi-24,25-(OH)₂D3. The method of the invention includes: preparing a standard curve equation, pre-treating a sample to be detected, and detecting the incoming sample. The pre-processing of the invention adopts the combination of protein precipitation, liquid-liquid extraction and derivation, so that the 11 vitamins D are extracted by one injection, and a pg-level sensitivity of the detection method is ensured at the same time. Moreover, the detection method of the invention also has the technical advantage of high accuracy.

### CROSS REFERENCE TO THE RELATED APPLICATIONS

This application is based upon and claims priority to Chinese Patent Application No. 202310982084.2, filed on Aug. 4, 2023, the entire contents of which are incorporated herein by reference.

### TECHNICAL FIELD

The present invention relates to the technical field of detection, and particularly to a Liquid Chromatography Tandem-mass Spectrometry (LC-MS/MS) analysis method for detecting 11 vitamins D in blood.

### BACKGROUND

Vitamin D is a lipid soluble vitamin, which plays an important role in bone homeostasis and immune regulation. Vitamin D deficiency is related to cardiovascular disease, kidney disease, cancer and other diseases. In addition, vitamin D supplementation can reduce the risk of fracture, especially in postmenopausal women and elderly men. There are two natural forms of vitamin D, comprising vitamin D3 (cholecalciferol) and vitamin D2 (ergocalciferol). Vitamin D3 can be synthesized by ultraviolet radiation, while vitamin D2 cannot be synthesized in vivo, so vitamin D2 can only be obtained from fortified foods and dietary supplements. Vitamin D is metabolized into 25-(OH)D by 25-hydroxylase in the liver, and 25-(OH)D is considered as the best biomarker to reflect the status of vitamin D in vivo. 25-(OH)D is metabolized into 1,25-(OH)₂D in kidney, which is called active vitamin D, and can promote bone health by maintaining calcium and phosphate levels in a steady state. In addition, 25-(OH)D can be metabolized into 24,25-(OH)₂D by 25-(OH)D-24-hydroxylase (CYP24), and its concentration is 2%-20% of the total 25-(OH)D. Previously, 24,25-(OH)₂D was considered as inactive vitamin D. With the further study of 24,25-(OH)₂D, it has been reported that serum 24,25-(OH)₂D can predict the level of vitamin D more than 25-(OH)D. In addition, a ratio between 24,25-(OH)₂D and 25-(OH)D is called a vitamin D metabolite ratio, and the vitamin D metabolite ratio has been suggested as a candidate biomarker of vitamin D status and a functional biomarker of bone health as the vitamin D metabolite ratio has no significant difference between races.

As is known, all major vitamin D metabolites can be epimerized at C3 position, and the amount of C3 epimers is higher in pregnant women and newborns. The C3 epimers and the vitamin D metabolites have the same precursor ions and product ions, which may lead to false positive results if they are not effectively separated. A study on infants and adults showed that 3-epi-25-(OH)D3 caused 90/(less than one year) and 3% (1 to 94 years old) patients to be wrongly diagnosed as vitamin D deficiency in the total concentration of 25-(OH)D. A previous study showed that if the epimers were not effectively separated by analytical methods, these interferences might account for 14%-55% of the total vitamin D level, so it is necessary to effectively separate the epimers in order to accurately monitor the vitamin level in vivo.

At present, a commonly used method to determine the content of the vitamin D in serum is by using LC-MS/MS. However, most of the reported literatures only detect 25-(OH)D, but not the key metabolites and epimers of the whole metabolic pathway of vitamin D, and most of the detection methods have some problems, such as complicated pre-treatment operation, large sample demand and long analysis time, which are not suitable for high-throughput sample detection.

In view of this, the present invention is provided.

### SUMMARY

In order to solve the above-mentioned technical problems, the present invention provides an LC-MS/MS analysis method for detecting 11 vitamins D in blood. Specifically, the 11 vitamins D and metabolites comprise: D2, D3, 1,25-(OH)₂D2, 1,25-(OH)₂D3, 25-(OH)D2, 25-(OH)D3, 3-epi-25-(OH)D2, 3-epi-25(OH)D3, 24,25-(OH)₂D2, 24,25-(OH)₂D3, and 3-epi-24,25-(OH)₂D3.

The present invention provides a method for simultaneously detecting 11 vitamins D by LC-MS/MS, wherein the 11 vitamin comprises D2, D3, 1,25-(OH)₂D2, 1,25-(OH)₂D3, 25-(OH)D2, 25-(OH)D3, 3-epi-25-(OH)D2, 3-epi-25-(OH)D3, 24,25-(OH)₂D2, 24,25-(OH)₂D3, and 3-epi-24,25-(OH)₂D3, and the method at least comprises the following steps of:
S1. preparing a standard curve equation, comprising: preparing an internal standard working liquid and a standard working liquid, preparing a sample solution for standard curve, and detecting the sample solution for standard curve by using an LC-MS/MS to obtain a standard curve equation for calculating contents of the 11 vitamins D in blood;
S2. pre-treating a sample to be detected, comprising: uniformly mixing the internal standard working liquid, the sample to be detected and a protein precipitant, extracting the mixture twice with an extractant to obtain a total supernatant, adding a derivatization reagent to part of the total supernatant, uniformly mixing, carrying out derivatization treatment, adding the remaining part of the total supernatant after blow-drying, blow-drying again, adding a reconstitution solution, uniformly mixing, and taking the supernatant as an incoming sample after centrifugation; the extractant being a mixed solution of normal hexane and methyl tert-butyl ether; and
S3. detecting the incoming sample, comprising: detecting the incoming sample by using the LC-MS/MS, and substituting a detection result of the incoming sample into the standard curve equation to obtain contents of 11 vitamins D in the sample to be detected.

Optionally, S1 comprises:
S11. respectively preparing an internal standard working liquid I, an internal standard working liquid II, a standard working liquid I and a standard working liquid U;
the internal standard working liquid I containing isotopic internal standards of D2, D3, 1,25-(OH)₂D2 and 1,25-(OH)₂D3; the internal standard working liquid H containing isotopic internal standards of 25-(OH)D2, 25-(OH)D3, 3-epi-25-(OH)D2, 3-epi-25-(OH)D3, 24,25-(OH)₂D2 and 24,25-(OH)₂D3; and the standard working liquid I containing standard solutions of D2, D3, 1,25-(OH)₂D2 and 1,25(OH)₂D3; and the standard working liquid II containing standard solutions of 25-(OH)D2, 25-(OH)D3, 3-epi-25-(OH)D2, 3-epi-25-(OH)D3, 24,25-(OH)₂D2, 24,25-(OH)₂D3 and 3-epi-24,25-(OH)₂D3;
S12. mixing the standard working liquid I and the internal standard working liquid I to prepare a standard curve working liquid with gradient concentration, blow-drying, adding the derivatization reagent for derivatization treatment, blow-drying the derived standard curve working liquid, respectively adding the standard working liquid TI and the internal standard working liquid II with corresponding concentrations according to the concentration gradient, adding the reconstitution solution, and mixing to obtain the sample solution for standard curve; and
S13. detecting the sample solution for standard curve by using the LC-MS/MS to obtain the standard curve equation.

Optionally, the derivatization reagent is selected from 50-500 μg/mL 4-Phenyl-3H-1,2,4-triazole-3,5(4H)-dione (PTAD) solution, and the derivatization lasts for 20-60 min; the reconstitution solution is selected from an aqueous solution containing 0.05%-0.2% formic acid by volume and 60%-100% methanol by volume; the protein precipitant is selected from methanol or anhydrous ethanol; and the extractant is a mixed solvent of normal hexane and methyl tert-butyl ether with a volume ratio of 2:1-4:1.

Optionally, in S12, a volume ratio of the standard working liquid I to the internal standard working liquid I is 1:1-2:1; a volume ratio of the internal standard working liquid I to the derivatization reagent is 1; 10-1:20; a volume ratio of the standard working liquid I to the standard working liquid II is 1:1; and a volume ratio of the standard working liquid II to the internal standard working liquid II and the reconstitution solution is 2:1:7.

Optionally, in S2, the internal standard working liquid I, the internal standard working liquid II, the sample to be detected and the protein precipitant are mixed, and a volume ratio of the internal standard working liquid I to the internal standard working liquid II is 1:1; a volume ratio of the sample to be detected to the protein precipitant is 1:1-2:1; a volume ratio of the supernatant for derivatization to the remaining part of supernatant is 1:1; and a volume ratio of the reconstitution solution to the sample to be detected is 1:1-1:2.

Optionally, in S2, the extracting twice with the extractant twice, comprises: a volume ratio of the sample to be detected to the extractant being 2:6.5-1:5; and a volume ratio of the extractant used during the second extraction to the extractant used during the first extraction being 0.8-1:1.

Compared with the prior art, the technical solutions provided by the embodiments of the present invention has the following advantages.

the detection method of the present invention detects the derivative products of D2, D3, 1,25-(OH)₂D2 and 1,25-(OH)₂D3, and the other 7 vitamins can be directly detected without derivatization, thus ensuring that th11 vitamins D can be extracted with one injection, and ensuring the pg-level sensitivity of the detection method.

Because the substance to be detected in the present invention is endogenous, the preparation of the standard curve of the present invention does not add matrix, and meanwhile, a matrix-free effect is ensured, and a recovery rate is good.

In preferred embodiments, the chromatographic conditions of the present invention can ensure that the 11 vitamins D have good chromatographic resolution, ensure the absolute separation of epimers and isomers, and meanwhile, the derivative products will not interfere with the separation of non-derivatives.

In another preferred technical solution, a standard dissolving reagent can ensure the solubility and storage stability of the standard.

### BRIEF DESCRIPTION OF THE DRAWINGS

FIG. 1 is a chromatogram of 1,25-(OH)₂D3 and 1,25-(OH)₂D2, and peaks from front to back are 1,25-(OH)₂D3 and 1,25-(OH)₂D2 in sequence;

FIG. 2 is a chromatogram of 24,25-(OH)₂D3, 3-epi-24,25-(OH)₂D3 and 24,25-(OH)₂D2, and peaks from front to back are 24,25-(OH)₂D3, 3-epi-24,25-(OH)₂D3 and 24,25-(OH)₂D2 in sequence;

FIG. 3 is a chromatogram of 25-(OH)D3 and 3-epi-25-(OH)D3, and peaks from front to back are 25-(OH)D3 and 3-epi-25-(OH)D3 in sequence;

FIG. 4 is a chromatogram of 25-(OH)D2 and 3-epi-25-(OH)D2, and peaks from front to back are 25-(OH)D2 and 3-epi-25-(OH)D2 in sequence;

FIG. 5 is a chromatogram of D3 and D2, and peaks from front to back are D3 and D2 in sequence;

FIGS. 6A-6H are chromatograms of 11 vitamins D in a sample, wherein:

Reference numeral 1 is a chromatographic peak of 25-(OH)D2, with a retention time of 5.92 min; reference numeral 2 is a chromatographic peak of 3-epi-25-(OH)D2, with a retention time of 6.09 min; reference numeral 3 is a chromatographic peak of 25-(OH)D3, with a retention time of 5.64 min; reference numeral 4 is a chromatographic peak of 3-epi-25-(OH)D3, with a retention time of 5.92 min; reference numeral 5 is a chromatographic peak of 24,25-(OH)₂D3, with a retention time of 3.98 min; reference numeral 6 is a chromatographic peak of 3-epi-24,25-(OH)₂D3, with a retention time of 4.15 min; reference numeral 7 is a chromatographic peak of 24,25-(OH)₂D2, with a retention time of 4.45 min; reference numeral 8 is a chromatographic peak of D3, with a retention time of 6.82 min; reference numeral 9 is a chromatographic peak of D2, with a retention time of 6.82 min; reference numeral 10 is a chromatographic peak of 1,25-(OH)₂D3, with a retention time of 3.42 min; and reference numeral 11 is a chromatographic peak of 1,25-(OH)₂D2, with a retention time of 3.64 min;

FIGS. 7A-7H arechromatograms of 11 vitamins D in a pure product; wherein, FIG. 7A is a chromatographic peak of 1,25-(OH)₂D3, FIG. 7B is a chromatographic peak of 1,25-(OH)₂D2, FIG. 7C is a chromatographic peak of 24,25-(OH)₂D3 and a chromatographic peak of 3-epi-24,25-(OH)₂D3, FIG. 7D is a chromatographic peak of 24,25-(OH)₂D2, FIG. 7E is a chromatographic peak of 25-(OH)D3 and 3-epi-25-(OH)D3, FIG. 7F is a chromatographic peak of 25-(OH)D2 and 3-epi-25-(OH)D2, FIG. 7G is a chromatographic peak of D3, and FIG. 7H is a chromatographic peak of D2;

FIG. 8 is a comparison diagram of pre-treatment effects of different protein precipitants;

FIG. 9 is a comparison diagram of extraction effects of different extractants;

FIG. 10 is a comparison diagram of experimental results at different derivatization reagent concentrations;

FIG. 11 is a comparison diagram of experimental results at different derivation times;

FIG. 12 is a chromatogram of D1 in Embodiment 5;

FIG. 13 is a chromatogram of D2 in Embodiment 5:

FIG. 14 is a chromatogram of D3 in Embodiment 5;

FIG. 15 is a chromatogram of D4 in Embodiment 5; and

FIG. 16 to FIG. 18 are chromatograms of D5 in Embodiment 5.

### DETAILED DESCRIPTION OF THE EMBODIMENTS

In order to better understand the above objects, features and advantages of the present invention, the solutions of the present invention will be further described below. It should be noted that, in case of no conflict, the embodiments in the present invention and the features in the embodiments may be combined with each other. In the following description, many specific details are set forth in order to fully understand the present invention, but the present invention may be implemented in other ways different from those described herein. Obviously, the embodiments described in the specification are merely a part of, rather than all of, the embodiments of the present invention.

The embodiments of the present invention provide a method for simultaneously detecting 11 vitamins D by LC-MS/MS. The 11 vitamins D comprise D2, D3, 1,25-(OH)₂D2, 1,25-(OH)₂D3, 25-(OH)D2, 25-(OH)D3, 3-epi-25-(OH)D2, 3-epi-25-(OH)D3, 24,25-(OH)₂D2, 24,25-(OH)₂D3, and 3-epi-24,25-(OH)₂D3, and the method at least comprises the following steps of:
S1. preparing a standard curve equation, comprising:
preparing an internal standard working liquid and a standard working liquid, preparing a sample solution for standard curve which is used for on-machine detection to prepare a standard curve equation, and detecting the sample solution for standard curve by using an LC-MS/MS to obtain a standard curve equation for calculating contents of the 11 vitamins D in blood;
S2. pre-treating a sample to be detected, comprising:
mixing the internal standard working liquid, the sample to be detected and a protein precipitant, extracting the mixture twice with an extractant to obtain a total supernatant, adding a derivatization reagent to part of the total supernatant, carrying out derivatization treatment, adding the remaining part of the total supernatant after blow-drying, blow-drying again, adding a reconstitution solution, uniformly mixing, and taking the supernatant as an incoming sample after centrifugation; the extractant being a mixed solution of normal hexane and methyl tert-butyl ether; and
S3. detecting the incoming sample, comprising:
detecting the incoming sample by using the LC-MS/MS, and substituting a detection result of the incoming sample into the standard curve equation to obtain contents of 11 vitamins D in the sample to be detected.

The sample to be detected in the embodiments of the present invention is a serum sample or a plasma sample, and the derivative products of D2, D3, 1,25-(OH)₂D2 and 1,25-(OH)₂D3 are detected by ingenious design of the pre-treatment steps in the embodiments of the present invention, and the other 7 vitamins D can be directly detected without derivatization, so that the 11 vitamins D can be simultaneously detected by one injection. The detection process is simple and fast, the experimental cost is reduced, and the analysis time is short, which is more conducive to the detection of large-volume samples.

In the research process of the embodiments of the present invention, it is found that if D2 and D3 are directly detected without derivatization, a matrix inhibition effect is too strong, and the pg-level sensitivity of the detection method cannot be guaranteed. Meanwhile, as 1,25-(OH)₂D2 and 1,25-(OH)₂D3 have low contents in blood, and are not easy to ionize, a response value during detection is low, and the pg-level sensitivity of the detection method cannot be guaranteed.

Therefore, the embodiments of the present invention choose to detect the derivative products of these four substances, so as to ensure that the detection sensitivity of D2, D3, 1,25-(OH)₂D2 and 1,25-(OH)₂D3 can reach the pg-level.

The reason why the derivative products of all substances are not completely detected in the present invention is that the analytical method needs to separate three groups of epimers, which are 25-(OH)D2, 3-epi-25-(OH)D2, 25-(OH)D3, 3-epi-25-(OH)D3, 24,25-(OH)₂D3, and 3-epi-24,25-(OH)₂D3. After derivatization, a polarity difference of the three groups of epimers becomes smaller, and under the existing gradient elution conditions, the three groups of epimers cannot achieve baseline separation, so the pre-treatment without derivatization is adopted in the present invention to ensure the separation of the three groups of epimers.
S1 comprises:
S11. respectively preparing an internal standard working liquid I, an internal standard working liquid H, a standard working liquid I and a standard working liquid U;
the internal standard working liquid I containing isotopic internal standards of D2, D3, 1,25-(OH)₂D2 and 1,25-(OH)₂D3;
the internal standard working liquid II containing isotopic internal standards of 25-(OH)D2, 25-(OH)D3, 3-epi-25-(OH)D2, 3-epi-25-(OH)D3, 24,25-(OH)₂D2 and 24,25-(OH)₂D3;
the standard working liquid I containing standard solutions of D2, D3, 1,25-(OH)₂D2 and 1,25-(OH)₂D3; and
the standard working liquid II containing standard solutions of 25-(OH)D2, 25-(OH)D3, 3-epi-25-(OH)D2, 3-epi-25-(OH)D3, 24,25-(OH)₂D2, 24,25-(OH)₂D3 and 3-epi-24,25-(OH)₂D3;
S12. mixing the standard working liquid I and the internal standard working liquid I to prepare a standard curve working liquid with gradient concentration, blow-drying, adding the derivatization reagent for derivatization treatment, blow-drying the derived standard curve working liquid, respectively adding the standard working liquid U and the internal standard working liquid H with corresponding concentrations according to the concentration gradient, adding the reconstitution solution, and mixing to obtain the sample solution for standard curve; and
S13. detecting the sample solution for standard curve by using the LC-MS/MS to obtain the standard curve equation.

Specifically, preparation methods of the solutions used in the embodiments of the present invention are as follows:
the preparation method of the standard working liquid I is: dissolving D2 and D3 solid standards with anhydrous ethanol or methanol to obtain a mother liquid, mixing the mother liquid with 1,25-(OH)₂D2 and 1,25-(OH)₂D3 liquid standards with known concentrations, and continuously diluting with anhydrous ethanol or methanol to obtain the standard working liquid I; and
a concentration gradient of the standard working liquid I contains at least 3 gradient concentrations, and preferably 5-10 gradient concentrations, and further preferably 8 gradient concentrations.

A preparation method of the standard working liquid 11 is: dissolving 24,25-(OH)₂D2 solid standard with 70%-100% methanol aqueous solution or/70%-100% ethanol aqueous solution by volume to obtain a mother liquid, and mixing the mother liquid with 25-(OH)D2, 25-(OH)D3, 3-epi-25-(OH)D2, 3-epi-25-(OH)D3, 24,25-(OH)₂D3 and 3-epi-24,25-(OH)₂D3 with known concentrations, and continuously diluting with 70%6-100% methanol aqueous solution or 70%-100% ethanol aqueous solution by volume to obtain the standard working liquid II; and
a concentration gradient of the standard working liquid II contains at least 3 gradient concentrations, and preferably 5-10 gradient concentrations, and further preferably 8 gradient concentrations.

In the preparation process of the standard working liquids, different diluents are selected according to different properties of the standards, thus ensuring the solubility and storage stability of the standards.

The preparation method of the internal standard working liquid I is: diluting commercially available liquid standards of isotopic internal standards of D2, D3, 1,25-(OH)₂D2 and 1,25-(OH)₂D3 with anhydrous ethanol or methanol to obtain the internal standard working liquid I; and
the preparation method of the internal standard working liquid H is: dissolving solid standards of isotopic internal standards of 25-(OH)D3, 24,25-(OH)₂D3 and 24,25-(OH)₂D2 with 70%-400% methanol aqueous solution or 70%-100% ethanol aqueous solution by volume to obtain a mother liquid, and mixing the mother liquid with liquid standards of isotopic internal standards of 25-(OH)D2, 3-epi-25-(OH)D2 and 3-epi-25-(OH)D3 with known concentrations, and diluting with 70%-100% methanol aqueous solution or 70%/O-100% ethanol aqueous solution by volume to obtain the internal standard working liquid II.

In the preparation process of the internal standard working liquids, different diluents are selected according to different properties of the internal standards, thus ensuring the solubility and storage stability of the internal standards.

As an improvement of the embodiments of the present invention, the derivatization reagent is selected from 50-500 μg/mL PTAD solution, and the concentration of the PTAD solution is preferably 100-200 μg/mL, more preferably 100-150 pg/mL, and may specifically be 100 μg/mL, 120 μg/mL, 125 μg/mL, 130 pg/mL, 135 μg/mL or 140 μg/mL; and more further preferably 125 μg/mL. The selection of this derivatization reagent can better eliminate a matrix effect in the detection of D3 and D2 samples, and greatly improve the detection sensitivity of 1,25-(OH)₂D2 and 1,25-(OH)₂D3. The derivatization may last for 20-60 min, and may last for 30 min specifically.

As an improvement of the embodiments of the present invention, the reconstitution solution is selected from an aqueous solution containing 0.05%-0.2% formic acid by volume and 60%-100% methanol by volume; further preferably, the reconstitution solution is selected from an aqueous solution containing 0.08%-0.15% formic acid by volume and 65%-75% methanol by volume; and more preferably, the reconstitution solution is selected from an aqueous solution containing 0.1% formic acid by volume and 70% methanol by volume. Using the reconstitution solution can ensure the solubility of the 11 vitamins D and minimize the matrix effect.

As an improvement of the embodiments of the present invention, the protein precipitant is selected from methanol or anhydrous ethanol, which can ensure the subsequent extraction effect of the 11 vitamins D.

As an improvement of the embodiments of the present invention, the extractant is a mixed solvent of normal hexane and methyl tert-butyl ether with a volume ratio of 2:1-4:1, may further be a mixed solvent of the n-hexane to the methyl tert-butyl ether with a volume ratio of 2.5:1, 3:1, 3.5:1 or 4:1, and more preferably, a mixed solvent of the volume ratio of the n-hexane and the methyl tert-butyl ether with a volume ratio of 4:1. Screening experiments show that the extraction effect can be improved and the pg-level sensitivity of endogenous substances detection can be ensured by using the extractant for twice extraction.

As an improvement of the embodiments of the present invention, in S12, a volume ratio of the standard working liquid I to the internal standard working liquid I is 1:1-2:1; a volume ratio of the standard working liquid I to the derivatization reagent is 1:10-1:20; a volume ratio of the standard working liquid I to the standard working liquid II is 1:1; and a volume ratio of the standard working liquid II to the internal standard working liquid 11 and the reconstitution solution is 2:1:7.

As an improvement of the embodiments of the present invention, the conditions of S12 are as follows: the derivatization reagent is added, and after vortex mixing at a rotating speed of 1,500-2,500 rpm for 30 s to 1 min, derivatization treatment is carried out, and after adding the reconstitution solution, vortex mixing is carried out at a rotating speed of 1,500-2,500 rpm for 1-3 min to obtain the sample solution for standard curve.

As one specific embodiment of the embodiments of the present invention, the specific conditions of S12 are as follows: the derivatization reagent is added, and after vortex mixing at a rotating speed of 2,000 rpm for 30 s, derivatization treatment is carried out, and after adding the reconstitution solution, vortex mixing is carried out at a rotating speed of 2,000 rpm for 1 min to obtain the sample solution for standard curve.

As an improvement of the embodiments of the present invention, in S12, the standard working liquid 1 and the internal standard working liquid 1 are prepared into the standard curve working liquid with gradient concentration by using methanol/anhydrous ethanol.

As one specific embodiment of the embodiments of the present invention, 20 μL of the standard working liquid I and 10 μL of the internal standard working liquid I with gradient concentration are taken to prepare 8 standard curve working liquids with different concentrations.

The standard curve working liquid is blow-dried by nitrogen, added with 200 μL of PTAD with a concentration of 125 μg/mL, subjected to vortex mixing at a rotating speed of 2,000 rpm for 30 s, derived for 30 min, the derived standard substance working liquid is blow-dried in nitrogen, 20 μL of the standard working liquid II and 10 μL of the internal standard working liquid II by using a pipette, added into a centrifugal tube after blow-drying by nitrogen, added with 70 μL of the reconstitution solution, subjected to vortex mixing at a rotating speed of 2,000 rpm for 1 min to obtain the sample solution for standard curve for on-machine detection.

As an improvement of the embodiments of the present invention, in S2, the internal standard working liquid I, the internal standard working liquid II, the sample to be detected and the protein precipitant are mixed, and a volume ratio of the internal standard working liquid I to the internal standard working liquid II is 1:1; a volume ratio of the sample to be detected to the protein precipitant is 1:1-2:1; a volume ratio of the supernatant for derivatization to the remaining part of supernatant is 1:1-9:10; and a volume ratio of the reconstitution solution to the sample to be detected is 1:1-1:2.

As an improvement of the embodiments of the present invention, the internal standard working liquid I, the internal standard working liquid 11, the sample to be detected and the protein precipitant are subjected to vortex mixing at a rotating speed of 1,500-2,500 rpm for 3-5 min, extracted twice with the extractant to obtain the total supernatant, the derivatization reagent is added to part of the total supernatant, and derivatization treatment is carried out after vortex mixing at a rotating speed of 1,500-2,500 rpm for 30 s to 1 min, the remaining part of the total supernatant is added after blow-drying, then the mixture is blow-dried again, added with the reconstitution solution, subjected to vortex mixing at a rotating speed of 1,500-2,500 rpm for 1-3 min, centrifuged at a rotating speed of 12,000-14,000 rpm for 5-10 min, and the supernatant is taken as the incoming sample.

As a specific embodiment of S2 of the embodiments of the present invention, the specific conditions of S2 are as follows: the internal standard working liquid 1, the internal standard working liquid II, the sample to be detected and the protein precipitant are subjected to vortex mixing at a rotating speed of 2,000 rpm for 3 min, extracted twice with the extractant to obtain the total supernatant, the derivatization reagent is added to part of the total supernatant, and derivatization treatment is carried out after vortex mixing at a rotating speed of 2,000 rpm for 30 s, the remaining part of the total supernatant is added after blow-drying, then the mixture is blow-dried again, added with the reconstitution solution, subjected to vortex mixing at a rotating speed of 2,000 rpm for 1 min, centrifuged at a rotating speed of 14,000 rpm for 5 min, and the supernatant is taken as the incoming sample.

As an improvement of the embodiments of the present invention, in S2, the extractant is used for extraction twice, during the first extraction, a volume ratio of the sample to be detected to the extractant is 2:6.5-1:5; and a volume ratio of the extractant used during the second extraction to the extractant used during the first extraction is 0.8-1:1.

The specific conditions of the extraction in S2 are as follows: during the first extraction, the internal standard working liquid, the sample to be detected and the protein precipitant are mixed uniformly, and then the extractant is added, and the mixture is subjected to vortex mixing at a rotating speed of 1,500-2,500 rpm for 3-5 min, and centrifuged at a rotating speed of 12,000-14,000 rpm for 5-10 min, and the supernatant is taken as supernatant I; the extractant is added into the centrifuged precipitate, subjected to vortex mixing at a rotating speed of 1,500-2,500 rpm for 3-5 min, centrifuged at a rotating speed of 12,000-14,000 rpm for 5-10 min, the supernatant is taken as a supernatant U, and the supernatant I and the supernatant II are combined to obtain the total supernatant.

As a specific embodiment of the extraction method according to the embodiments of the present invention, during the first extraction, the internal standard working liquid, the sample to be detected and the protein precipitant are mixed uniformly, and then the extractant is added, and the mixture is subjected to vortex mixing at a rotating speed of 2,000 rpm for 5 min, and centrifuged at a rotating speed of 14,000 rpm for 10 min, and the supernatant is taken as supernatant I; the extractant is added into the centrifuged precipitate, and the mixture is subjected to vortex mixing at a rotating speed of 2,000 rpm for 5 min, centrifuged at a rotating speed of 14,000 rpm for 5-10 min, the supernatant is taken as a supernatant II, and the supernatant I and the supernatant II are combined to obtain the total supernatant.

As one specific embodiment of the embodiments of the present invention, S2 comprises: taking 10 μL of internal standard working liquid I and 10 μL of internal standard working liquid U, adding 200 μL of serum sample to be detected, adding 100 μL of protein precipitant, carrying out vortex mixing at a rotating speed of 2,000 rpm for 3 min, adding 650 μL of extractant, carrying out vortex mixing at a rotating speed of 2,000 rpm for 5 min, centrifuging at a rotating speed of 14,000 rpm for 10 min, taking 600 μL of supernatant I, adding 650 μL of extractant to the centrifuged precipitate, carrying out vortex mixing at a rotating speed of 2,000 rpm for 5 min, centrifuging at a rotating speed of 14,000 rpm for 10 min, taking the supernatant as the supernatant 11, and combining the supernatant I and the supernatant II to obtain the total supernatant. 600 μL of the total supernatant was put into a 1.5 mL centrifuge tube, added with 200 μL of PTAD with a concentration of 125 μg/mL, subjected to vortex mixing at a rotating speed of 2,000 rpm for 30 s, derivatized for 30 min, and then blow-dried by nitrogen. The remaining 600 μL supernatant of the total supernatant is added into the derivatized centrifuge tube, blow-dried with nitrogen, added with 100 μL of reconstitution solution, subjected to vortex mixing at a rotating speed of 2,000 rpm for 1 min, centrifuged at a rotating speed of 14,000 rpm for 5 min, and 90 μL supernatant is taken as the solution to be detected.

The five groups of isomers in the substances to be detected in the present invention are: 25-(OH)D2 and 3-epi-25-(OH)D2, 25-(OH)D3 and 3-epi-25-(OH)D3, 24,25-(OH)₂D3 and 3-epi-24,25-(OH)₂D3, 24,25-(OH)₂D3 and 1,25-(OH)₂D3 as well as 24,25-(OH)₂D2 and 1,25-(OH)₂D2. Therefore, it is necessary to realize chromatographic separation of the above substances. Therefore, high-performance liquid phase conditions are studied in the embodiments of the present invention, thereby realizing the complete separation of all substances before and after derivatization, avoiding ion crosstalk and improving the detection accuracy.

As an improvement of the embodiments of the present invention, the high-performance liquid chromatography conditions when using the LC-MS/MS for detection are as follows: the chromatographic column can be pentafluorophenyl chromatographic column with a particle size of 2.6 μm; Specifically, Phenomenex Kinetex F5 100A chromatographic column (2.6 μm, 3.0 mm×50 mm) may be used.

The mobile phases may be as follows: phase A is an aqueous solution containing 0.05%-0.2% formic acid and 1 mM-5 mM ammonium formate or ammonium acetate; and phase B is a methanol solution containing 0.05%-0.2% formic acid and 1 mM-5 mM ammonium formate or ammonium acetate. Further preferably, the phase A is an aqueous solution containing 0.1% formic acid and 1 mM ammonium formate; and phase B is a methanol solution containing 0.1% formic acid and 1 mM ammonium formate.
a flow rate is 0.25-0.35 mL/min, preferably 0.3 mL/min; a column temperature is 25-35° C., preferably 30° C.; a sample volume is 10-20 μL, preferably 20 μL; and an analysis time is 8 min.

The gradient elution conditions are as follows:
at 0-1.50 min, the phase A changes from a concentration A1 to a concentration A2 at a constant speed, and the phase B changes from a concentration B1 to a concentration B2 at a constant speed;
at 1.50-2.50 min, the phase A adopts the concentration A2, and the phase B adopts the concentration B2;
at 2.50-3.00 min, the phase A changes from the concentration A2 to a concentration A3 at a constant speed, and the phase B changes from the concentration B2 to a concentration B3 at a constant speed;
at 3.00-5.00 min, the phase A adopts the concentration A3, and the phase B adopts the concentration B3;
at 5.00-5.10 min, the phase A changes from the concentration A3 to a concentration A4 at a constant speed, and the phase B changes from the concentration B3 to a concentration B4 at a constant speed;
at 5.10-6.50 min, the phase A adopts the concentration A4, and the phase B adopts the concentration B4;
at 6.51-8.00 min, the phase A adopts a concentration A5, and the phase B adopts a concentration B5;
the concentration A1 is selected from 40%-30%, the concentration B1 is selected from 60%-70%, and the concentration A1+the concentration B1=100%;
the concentration A2 is selected from 28%-24%, the concentration B2 is selected from 72%-76%, and the concentration A2+the concentration B2=100%;
the concentration A3 is selected from 22%-15%, the concentration B3 is selected from 78%-85%, and the concentration A3+the concentration B3=100%;
the concentration A4 is selected from 10%-0, the concentration B4 is selected from 90%-100%, and the concentration A4+the concentration B4=100%; and
the concentration A5 is selected from 40%-30%, the concentration B5 is selected from 60%-70%, and the concentration A5+the concentration B5=100%.

Preferably, the concentration A1 is selected from 38%-32%, the concentration B1 is selected from 62%-68%, and the concentration A1+the concentration B1=100%;
the concentration A2 is selected from 28%-24%, the concentration B2 is selected from 72%-76%, and the concentration A2+the concentration B2=100%;
the concentration A3 is selected from 22%-18%, the concentration B3 is selected from 78%-82%, and the concentration A3+the concentration B3=100%;
the concentration A4 is selected from 5%-0, the concentration B4 is selected from 95%-1009%, and the concentration A4+the concentration B4=100%; and
the concentration A5 is selected from 38%-32%, the concentration B5 is selected from 62%-68%, and the concentration A5+the concentration B5=1009%.

Further preferably, the gradient elution conditions are as shown in Table 1.

TABLE 1

| Time/min | Phase A | Phase B |
|---|---|---|
| 0.00 | 35 | 65 |
| 1.50 | 26 | 74 |
| 2.50 | 26 | 74 |
| 3.00 | 20 | 80 |
| 5.00 | 20 | 80 |
| 5.10 | 0 | 100 |
| 6.50 | 0 | 100 |
| 6.51 | 35 | 65 |
| 8.00 | 35 | 65 |

As an improvement of the embodiments of the present invention, the mass spectrum conditions when using the LC-MS/MS for detection are as follows: using electrospray ion source (ESI) and positive ion mode for multi-reaction monitoring under ionspray voltage: 5,000 V-5,500 V, preferably 5,500 V; ion source temperature: 300-400° C., preferably 350° C.; atomizing gas: 45-55 psi, preferably 50 psi; auxiliary gas: 25-35 psi, preferably 30 psi; curtain gas: 20-25 psi, preferably 20 psi; and collision gas: 8-10 psi, preferably 9 psi.

Ion pair parameters are as shown in Table 2.

TABLE 2

| | Quantitative | | Qualitative | | | | | | |
|---|---|---|---|---|---|---|---|---|---|
| | ion pair | | ion pair | | Retention | | | | |
| | Parent | Daughter | Parent | Daughter | time | | | | |
| Substance name | ion | ion | ion | ion | (min) | DP | CE | EP | CXP |
| 24,25-(OH)₂D3-d6 | 423.2 | 387.3 | / | / | 3.98 | 76 | 16 | 10 | 10 |
| 24,25-(OH)₂D2-d3 | 396.4 | 246.4 | / | / | 4.45 | 45 | 18 | 10 | 10 |
| 3-epi-25-(OH)D3-d3 | 404.3 | 368.1 | / | / | 5.92 | 60 | 19 | 10 | 10 |
| 25-(OH)D2-d3 | 416.2 | 340.2 | / | / | 5.92 | 60 | 16 | 10 | 10 |
| 25-(OH)D3-d6 | 407.3 | 371.1 | / | / | 5.64 | 60 | 19 | 10 | 10 |
| 25-(OH)D2 | 413.2 | 337.2 | 413.2 | 355.3 | 5.92 | 60 | 15 | 10 | 10 |
| 25-(OH)D3 | 401.3 | 365.1 | 401.3 | 383.3 | 5.64 | 60 | 19 | 10 | 10 |
| 3-epi-25-(OH)D2 | 413.2 | 337.2 | 413.2 | 355.3 | 6.09 | 60 | 15 | 10 | 10 |
| 3-epi-25-(OH)D3 | 401.3 | 365.1 | 401.3 | 355.3 | 5.92 | 60 | 19 | 10 | 10 |
| 24,25-(OH)₂D3 | 417.2 | 381.3 | 417.2 | 399.1 | 3.98 | 76 | 16 | 10 | 10 |
| 24,25-(OH)₂D2 | 393.4 | 243.4 | 393.4 | 268.1 | 4.45 | 45 | 18 | 10 | 10 |
| 3-epi-25-(OH)D2-C5 | 418.2 | 340.2 | / | / | 5.92 | 60 | 15 | 10 | 10 |
| 3-epi-24,25-(OH)₂D3 | 417.2 | 381.3 | 417.2 | 399.1 | 4.15 | 76 | 16 | 10 | 10 |
| D3 | 560.3 | 298.1 | 560.3 | 365.2 | 6.82 | 60 | 22 | 10 | 10 |
| D2 | 572.3 | 298.1 | 572.3 | 280.3 | 6.82 | 60 | 22 | 10 | 10 |
| D3-d3 | 563.3 | 301.1 | / | / | 6.82 | 60 | 22 | 10 | 10 |
| D2-d3 | 575.3 | 301.1 | / | / | 6.82 | 60 | 22 | 10 | 10 |
| 1,25-(OH)₂D3 | 574.3 | 314.3 | 574.3 | 243.8 | 3.42 | 55 | 26 | 10 | 10 |
| 1,25-(OH)₂D2 | 586.1 | 314.3 | 604.1 | 314.3 | 3.64 | 70 | 21 | 10 | 10 |
| 1,25-(OH)₂D3-C3 | 577.3 | 314.3 | / | / | 3.42 | 55 | 26 | 10 | 10 |
| 1,25-(OH)₂D2-C3 | 589.1 | 314.3 | / | / | 3.64 | 70 | 21 | 10 | 10 |

### Embodiment 1

The embodiment provided a liquid chromatogram analysis method for contents of 11 vitamins D, which comprised the following steps.

### (1) Preparation of Solution:

### 1. A Preparation Method of a Standard Working Liquid I was as Follows:

D3 and D2 standards were accurately weighed and dissolved with anhydrous ethanol to obtain mother liquids with corresponding concentrations. 1,25-(OH)₂D2 and 1,25-(OH)₂D3 were liquid standards in the market, and the mother liquid concentrations were known. A certain volume of the mother liquids of the above three vitamins were taken and diluted with anhydrous ethanol to obtain corresponding intermediate liquids. The specific mother liquid/intermediate liquid concentrations were shown in Table 3.

TABLE 3

| | | Intermediate |
|---|---|---|
| | Mother liquid | liquid concentration |
| Target | concentration (μg/mL) | (μg/mL) |
| D2 | 2219 | 20 |
| D3 | 3268 | 100 |
| 1,25-(OH)₂ | 5 | / |
| D2 | | |
| 1,25-(OH)₂ | 5 | 1.28 |
| D3 | | |

A certain volume of the mother liquids/intermediate liquids of the above four vitamins D were taken, and continuously diluted with anhydrous ethanol to obtain an L8 point of the standard working liquid I, as shown in Table 4.

TABLE 4

| | Mother | Volume of | | | Working |
|---|---|---|---|---|---|
| | liquid/ | mother | | | liquid con- |
| | intermediate | liquid/ | | | centration |
| | liquid | intermediate | | Diluent | at L8 |
| | concentration | liquid | | volume | point |
| Target | (μg/mL) | taken (mL) | Diluent | (mL) | (ng/ml) |
| D2 | 20 | 10 | Anhydrous | 951.2 | 200 |
| D3 | 100 | 16 | ethanol | | 1600 |
| 1,25-(OH)₂ | 5 | 12.8 | | | 64 |
| D2 | | | | | |
| 1,25-(OH)₂ | 1.28 | 10 | | | 12.8 |
| D3 | | | | | |

Preparation process of L7-L1 point (ng/mL): from L8 point, the standard working liquid I was obtained by gradual dilution with anhydrous ethanol, as shown in Table 5.

TABLE 5

| | Concentration of standard working liquid I (ng/mL) | | | | | | | |
|---|---|---|---|---|---|---|---|---|
| Target | L8 | L7 | L6 | L5 | L4 | L3 | L2 | L1 |
| D2 | 200 | 100 | 50 | 25 | 12.5 | 6.25 | 3.125 | 1.5625 |
| D3 | 1600 | 800 | 400 | 200 | 100 | 50 | 25 | 12.5 |
| 1,25-(OH)₂ | 64 | 32 | 16 | 8 | 4 | 2 | 1 | 0.5 |
| D2 | | | | | | | | |
| 1,25-(OH)₂ | 12.8 | 6.4 | 3.2 | 1.6 | 0.8 | 0.4 | 0.2 | 0.1 |
| D3 | | | | | | | | |

### 2. A Preparation Method of a Standard Working Liquid II was as Follows:

A certain amount of 24,25-(OH)₂D2 standard were accurately weighed and dissolved with 70% methanol water to obtain mother liquids with corresponding concentrations. The other six vitamins D were all liquid standards in the market with known concentrations. A certain volume of the mother liquids of the above five vitamins were taken and diluted with 70% a methanol to obtain corresponding intermediate liquids. The specific mother liquid/intermediate liquid concentrations were shown in Table 6.

TABLE 6

| | Mother liquid | Intermediate liquid |
|---|---|---|
| | concentration | concentration |
| Target | (μg/mL) | (μg/mL) |
| 25-(OH)D2 | 50 | / |
| 25-(OH)D3 | 100 | / |
| 3-epi-25-(OH)D2 | 107.4 | 16 |
| 3-epi-25-(OH)D3 | 50 | 32 |
| 24,25-(OH)₂D2 | 997.8 | 16 |
| 24,25-(OH)₂D3 | 107.7 | 10 |
| 3-epi-24,25-(OH)₂D3 | 99.8 | 10 |

A certain volume of the mother liquids/intermediate liquids shown in Table 6 were taken, and diluted with 70% methanol aqueous solution to obtain an L8 point of the standard working liquid II, which were specifically shown in Table 7.

TABLE 7

| | Mother | Volume of | | | Working |
|---|---|---|---|---|---|
| | liquid/inter- | mother | | | liquid con- |
| | mediate | liquid/inter- | | | centration |
| | liquid con- | mediate | | Diluent | at L8 |
| | centration | liquid | | volume | point |
| Target | (μg/mL) | taken (mL) | Diluent | (mL) | (ng/mL) |
| 25-(OH)D2 | 50 | 4 | Meth- | 948 | 200 |
| 25-(OH)D3 | 100 | 16 | anol: | | 1600 |
| 3-epi-25- | 16 | 5 | water = | | 80 |
| (OH)D2 | | | 7:3 | | |
| 3-epi-25- | 32 | 10 | | | 320 |
| (OH)D3 | | | | | |
| 24,25- | 16 | 5 | | | 80 |
| (OH)₂D2 | | | | | |
| 24,25- | 10 | 8 | | | 80 |
| (OH)₂D3 | | | | | |
| 3-epi- | 10 | 4 | | | 40 |
| 24,25- | | | | | |
| (OH)₂ | | | | | |
| D3 | | | | | |

Preparation process of L7-L1 point: from L8 point, the standard working liquid 11 was obtained by gradual dilution with 70% i methanol, as shown in Table 8.

TABLE 8

| | Concentration of standard working liquid II (ng/mL) | | | | | | | |
|---|---|---|---|---|---|---|---|---|
| Target | L8 | L7 | L6 | L5 | L4 | L3 | L2 | L1 |
| 25-(OH)D2 | 200 | 100 | 50 | 25 | 12.5 | 6.25 | 3.125 | 1.5625 |
| 25-(OH)D3 | 1600 | 800 | 400 | 200 | 100 | 50 | 25 | 12.5 |
| 3-epi-25-(OH)D2 | 80 | 40 | 20 | 10 | 5 | 2.5 | 1.25 | 0.625 |
| 3-epi-25-(OH)D3 | 320 | 160 | 80 | 40 | 20 | 10 | 5 | 2.5 |
| 24,25-(OH)₂D2 | 80 | 40 | 20 | 10 | 5 | 2.5 | 1.25 | 0.625 |
| 24,25-(OH)₂D3 | 80 | 40 | 20 | 10 | 5 | 2.5 | 1.25 | 0.625 |
| 3-epi-24,25-(OH)₂ | 40 | 20 | 10 | 5 | 2.5 | 1.25 | 0.625 | 0.3125 |
| D3 | | | | | | | | |

### 3. A Preparation Method of an Internal Standard Working Liquid I was as Follows:

D2, D3, 1,25-(OH)₂D2 and 1,25-(OH)₂D3 isotopic internal standards were all commercially available liquid standard standards with known concentrations. A certain volume of the internal standard mother liquids of the above four vitamins were taken and diluted with anhydrous ethanol to obtain corresponding internal standard intermediate liquids. The specific mother liquid/intermediate liquid concentrations were shown in Table 9.

TABLE 9

| Internal | Mother liquid concentration | Intermediate liquid |
|---|---|---|
| standard | (μg/mL) | concentration (μg/mL) |
| D2-d3 | 99.9 | 5 |
| D3-d3 | 100 | 10 |
| 1,25-(OH)₂D2- | 5.499 | 0.8 |
| C5 | | |
| 1,25-(OH)₂D3- | 5.336 | 0.2 |
| C5 | | |

The internal standard intermediate liquid shown in Table 9 was continuously diluted with anhydrous ethanol to obtain the internal standard working liquid 1, and the specific preparation process was shown in Table 10.

TABLE 10

| | | Volume of | | | Internal |
|---|---|---|---|---|---|
| | | mother | | | standard |
| | Intermediate | liquid/ | | | working |
| | liquid | intermediate | | Diluent | liquid con- |
| Internal | concentration | liquid | | volume | centration |
| standard | (μg/mL) | taken (mL) | Diluent | (mL) | (ng/mL) |
| D2 | 5 | 10 | Anhydrous | 950 | 50 |
| D3 | 10 | 20 | ethanol | | 200 |
| 1,25- | 0.8 | 10 | | | 8 |
| (OH)₂D2 | | | | | |
| 1,25- | 0.2 | 10 | | | 2 |
| (OH)₂D3 | | | | | |

### 4. A Preparation Method of an Internal Standard Working Liquid II was as Follows:

JA certain amount of 25-(OH)D3-d6, 24,25-(OH)₂D3-d3 and 24,25-(OH)₂D2-d6 standards were accurately weighed and dissolved with 70% i methanol to obtain mother liquids with corresponding concentrations. 25-(OH)D2-d3, 3-epi-25-(OH)D2-C5, 3-epi-25-(OH)D3-d3 were all commercially available liquid standard standards with known concentrations. A certain volume of the internal standard mother liquids of the above six vitamins were taken and diluted with 70% methanol to obtain corresponding internal standard intermediate liquids. The specific mother liquid/intermediate liquid concentrations were shown in Table 11.

TABLE 11

| | Mother liquid | Intermediate liquid |
|---|---|---|
| | concentration | concentration |
| Internal standard | (μg/mL) | (μg/mL) |
| 25-(OH)D2-d3 | 105.1 | 5 |
| 25-(OH)D3-d6 | 500 | 100 |
| 3-epi-25-(OH)D2-C5 | 50 | 2 |
| 3-epi-25-(OH)D3-d3 | 106.7 | 8 |
| 24,25-(OH)₂D2-d6 | 500 | 2 |
| 24,25-(OH)₂D3-d3 | 1000 | 2 |

The internal standard intermediate liquid shown in Table 11 was continuously diluted 70% methanol aqueous solution to obtain the internal standard working liquid 11, and the specific preparation process was shown in Table 12.

TABLE 12

| | | Volume of | | | |
|---|---|---|---|---|---|
| | | mother | | | |
| | Intermediate | liquid/ | | | Internal standard |
| | liquid | intermediate | | Diluent | working liquid |
| | concentration | liquid taken | | volume | concentration |
| Internal standard | (μg/mL) | (mL) | Diluent | (mL) | (ng/mL) |
| 25-(OH)D2-d3 | 5 | 10 | Methanol: | 870 | 50 |
| 25-(OH)D3-d6 | 100 | 80 | water = 7:3 | | 8000 |
| 3-epi-25-(OH)D2- | 2 | 10 | | | 20 |
| C5 | | | | | |
| 3-epi-25-(OH)D3- | 8 | 10 | | | 80 |
| d3 | | | | | |
| 24,25-(OH)₂D2-d6 | 2 | 10 | | | 20 |
| 24,25-(OH)₂D3-d3 | 2 | 10 | | | 20 |

The prepared standard working liquids and internal standard working liquids were marked with names and concentrations, and stored at −80° C., and valid for at least 3 months.

### (2) Calibration of Standard Curve Working Liquid

20 μL of standard working liquid I and 10 μL of internal standard working liquid I were respectively taken by a pipette and placed in a 1.5 mL centrifuge tube to prepare eight standard curve working liquids with different concentrations, blow-dried by nitrogen, added with 200 μL of PTAD with a concentration of 125 μg/mL, and subjected to vortex mixing at a rotating speed of 2,000 rpm for 30 s, and derived for 30 min. The derived standard working liquids were blow-dried by nitrogen. 20 μL of standard working liquid II and 10 μL of internal standard working liquid II were respectively taken by a pipette and placed in a centrifuge tube blow-dried by nitrogen, and then added with 70 μL of 70% methanol aqueous solution (0.1% formic acid), and subjected to vortex mixing at a rotating speed of 2,000 rpm for 1 min to obtain the sample solution for standard curve.

The sample solution for standard curve was detected by using an LC-MS/MS to obtain standard solution chromatograms of 11 vitamins D (D3, D2, 1,25-(OH)₂D3, 1,25-(OH)₂D2, 25-(OH)D3, 25-(OH)D2, 3-epi-25(OH)D3, 3-epi-25-(OH)D2, 24,25-(OH)₂D3, 24,25-(OH)₂D2 and 3-epi-24,25-(OH)₂D3) with eight different concentrations. Peak areas of the 11 vitamins D and the internal standards were obtained from the standard solution chromatograms of the above 11 vitamins D and the internal standards respectively. A ratio of the peak areas of the standard solutions of the 11 vitamins D with eight different concentrations to the peak area of the internal standards as an ordinate y of the standard curve equation, and taking a ratio of the concentration of the standard working liquid of the 11 vitamins D to the concentration of the internal standard working liquid as an abscissa x of the standard curve equation, the data of the eight different concentrations detected above were linearly regressed, the standard curve equation was fitted as y=a*x+b, and the linear equation coefficients a and b were obtained.

### (3) Treatment of Sample to be Detected

10 μL of internal standard working liquid I and 10 μL of internal standard working liquid II were respectively taken with a pipette first and then put into a 1.5 mL centrifuge tube, 200 μL of serum sample to be detected was taken by a pipette, added with 100 μL of methanol, subjected to vortex mixing at a rotating speed of 2,000 rpm for 3 min, added with 650 μL of n-hexane: methyl tert-butyl ether=4:1, subjected to vortex mixing at a rotating speed of 2,000 rpm for 5 min, and centrifuged at a rotating speed of 14,000 rpm for 10 min, 600 μL of supernatant I was added into a clean 1.5 mL centrifuge tube, and then 650 μL of n-hexane: methyl tert-butyl ether=4:1 was added into the 1.5 mL centrifuge tube, subjected to vortex mixing at a rotating speed of 2,000 rpm for 5 min, and centrifuged at a rotating speed of 14,000 rpm for 10 min to obtain a supernatant II. The supernatant I and the supernatant II were combined to obtain a total supernatant. 600 μL of the total supernatant was put into a 1.5 mL centrifuge tube, added with 200 μL of PTAD with a concentration of 125 μg/mL, subjected to vortex mixing at a rotating speed of 2,000 rpm for 30 s, derivatized for 30 min, and then blow-dried by nitrogen. The remaining 600 μL supernatant of the total supernatant was added into the derivatized centrifuge tube, blow-dried with nitrogen, added with 100 μL of reconstitution solution (water solution containing 0.1% formic acid and 70% methanol by volume) for redissolution, subjected to vortex mixing at a rotating speed of 2,000 rpm for 1 min, centrifuged at a rotating speed of 14,000 rpm for 5 min, and 90 μL supernatant was taken as the solution to be detected.

### (4) Detection of Incoming Sample

The incoming sample in the step (3) was detected by using an LC-MS/MS to obtain chromatograms of the 11 vitamins D of the incoming sample and the internal standard, and obtain peak areas of the 11 vitamins D and the internal standard from the chromatograms of the 11 vitamins D and the internal standard. Substituting a ratio y of the peak areas of the 11 vitamins D to the internal standard into a standard curve equation that y=a*x+b in the step (2), relative concentrations x of the 11 vitamins D in the incoming sample and the internal standard were obtained by calculation, and the working liquid concentration of the internal standard was known, so the concentrations of the 11 vitamins D in the blood sample to be detected were calculated.

Chromatographic column used for chromatographic analysis was phenomenex Kinetex F5 100A chromatographic column (2.6 μm, 3.0 mm×50 mm). A mobile phase consisted of water (0.1% formic acid+1 mM ammonium formate) and methanol (0.1% formic acid+1 mM ammonium formate), with gradient elution (as shown in Table 1) at a flow rate of 0.3 mL/min, a column temperature of 30° C., and a sample volume of 20 μL; and an analysis time was 8 min.

A mass spectrum detector was SCEIX AB6500 detector, adopting electrospray ion source (ESI), positive ion mode, multi-reaction monitoring (MRM), an ionspray voltage (IonSpray Voltage) of 5,500 V; an ion source temperature (TEM) of 350° C.; an atomizing gas (Gas1) of 50 psi; an auxiliary gas (Gas2) of 30 psi; a curtain gas (CUR) of 20 psi; and a collision gas of 9 psi. Ion pair parameters were shown in Table 2.

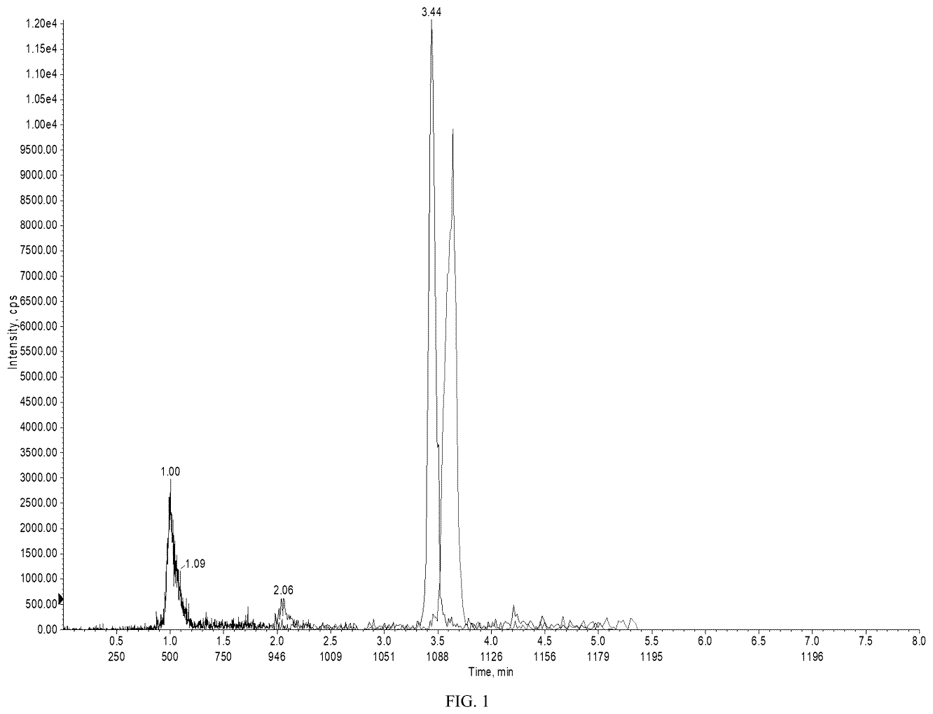

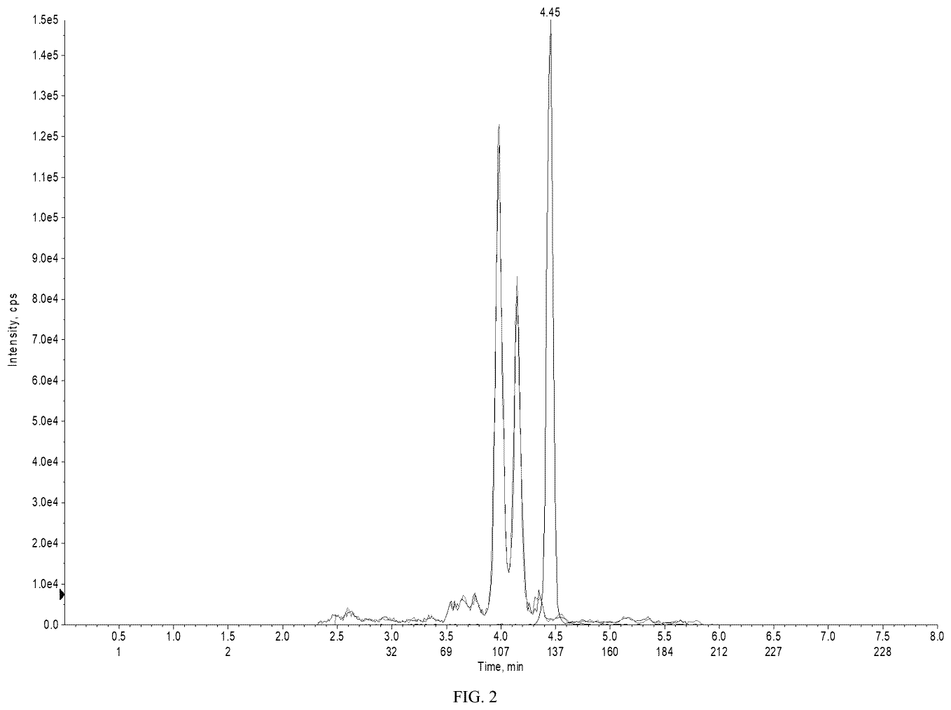

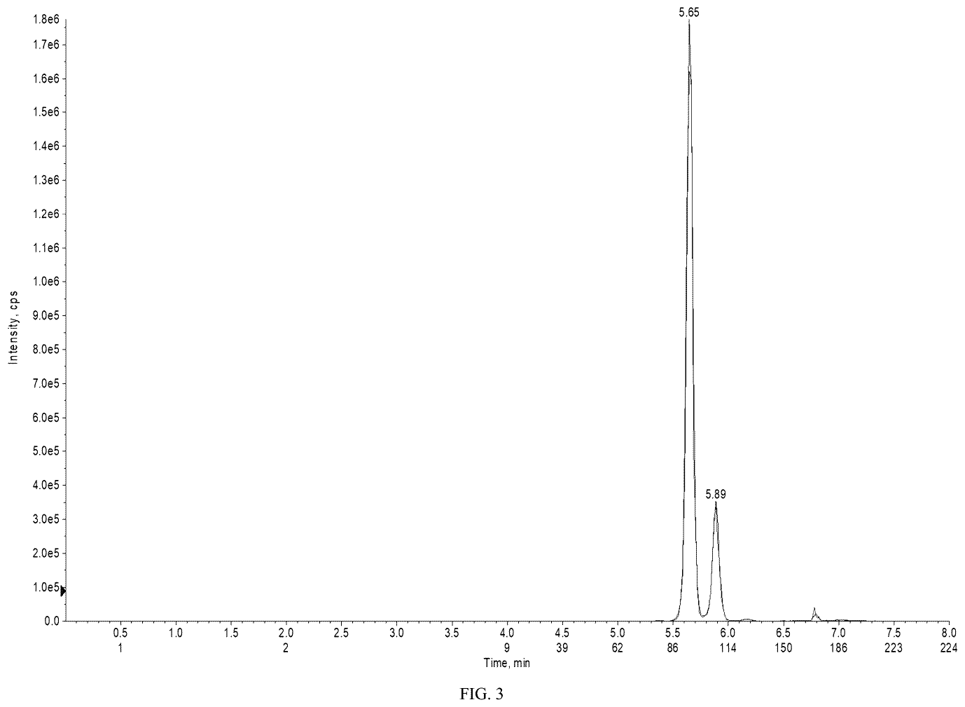

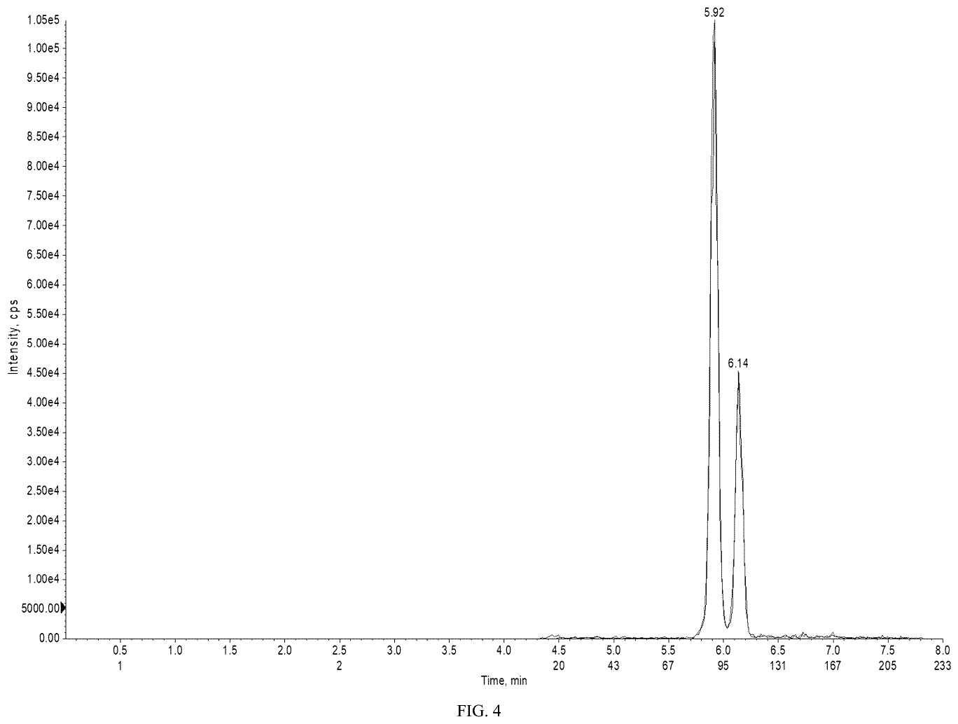

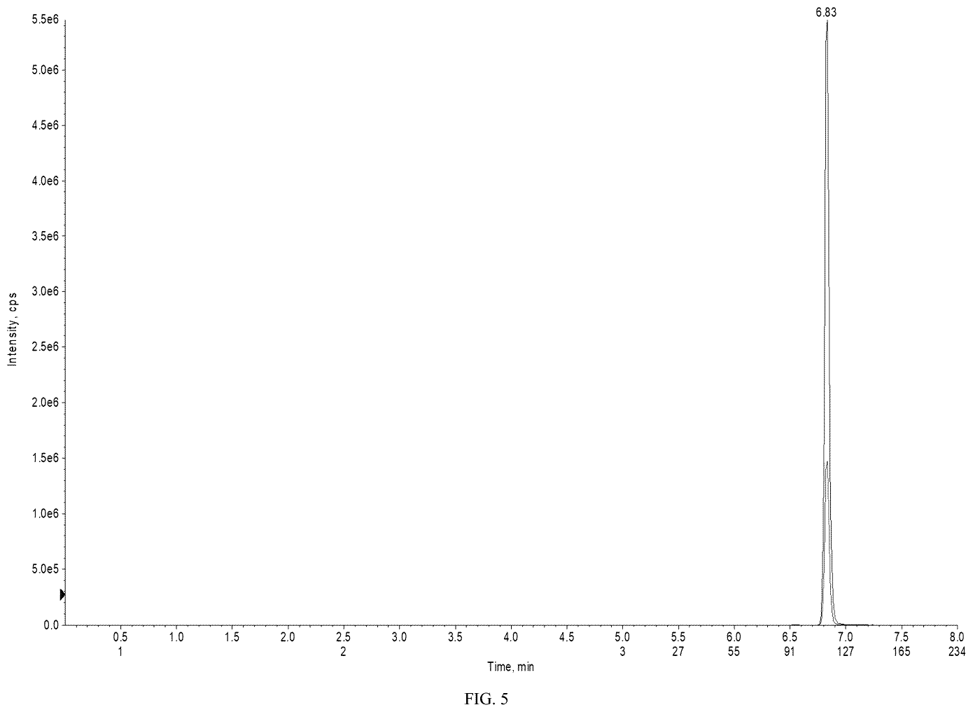

The obtained chromatograms were shown in FIG. 1 to FIGS. 6A-6H, wherein FIG. 1 was the chromatogram of 1,25-(OH)₂D3 and 1,25-(OH)₂D2, FIG. 2 was the chromatogram of 24,25-(OH)₂D3, 3-epi-24,25-(OH)₂D3 and 24,25-(OH)₂D2, FIG. 3 was the chromatogram of 25-(OH)D3 and 3-epi-25-(OH)D3, FIG. 4 was the chromatogram of 25-(OH)D2 and 3-epi-25-(OH)D2, and FIG. 5 was the chromatogram of D3 and D2. It could be seen from FIG. 2 to FIG. 4 that the three groups of epimers had all achieved chromatographic separation.

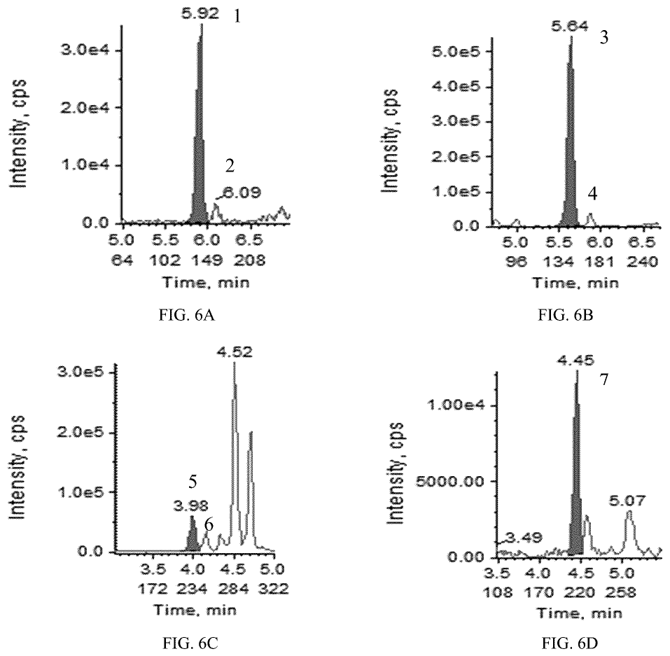

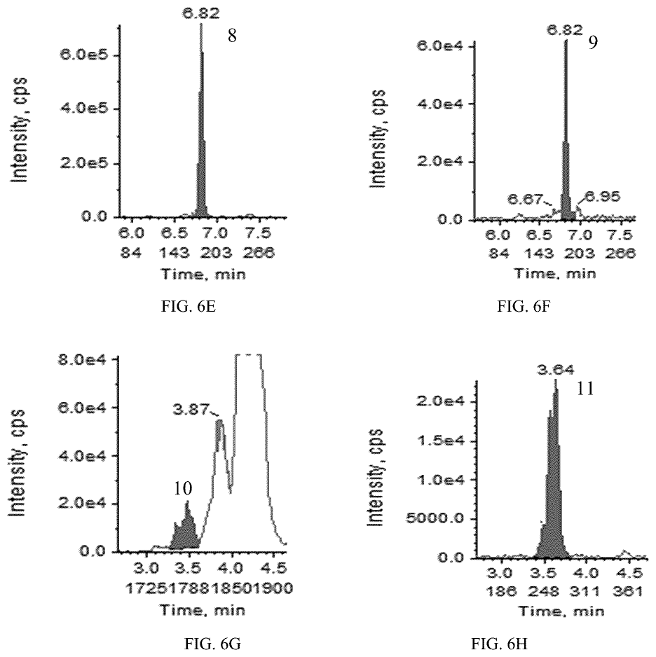

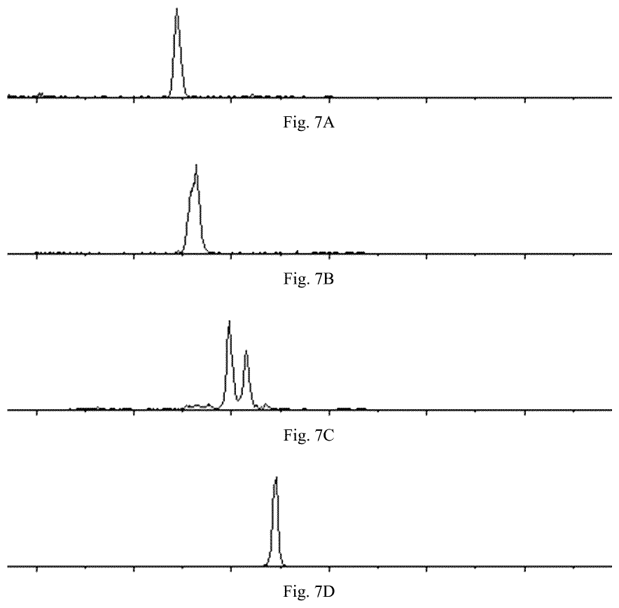

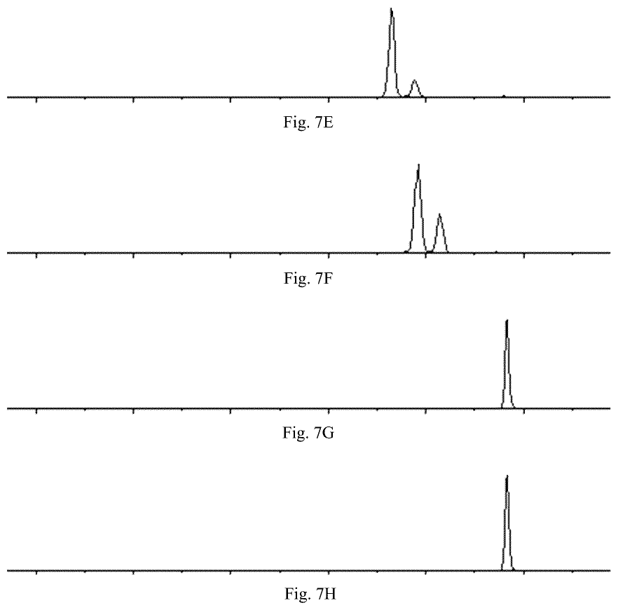

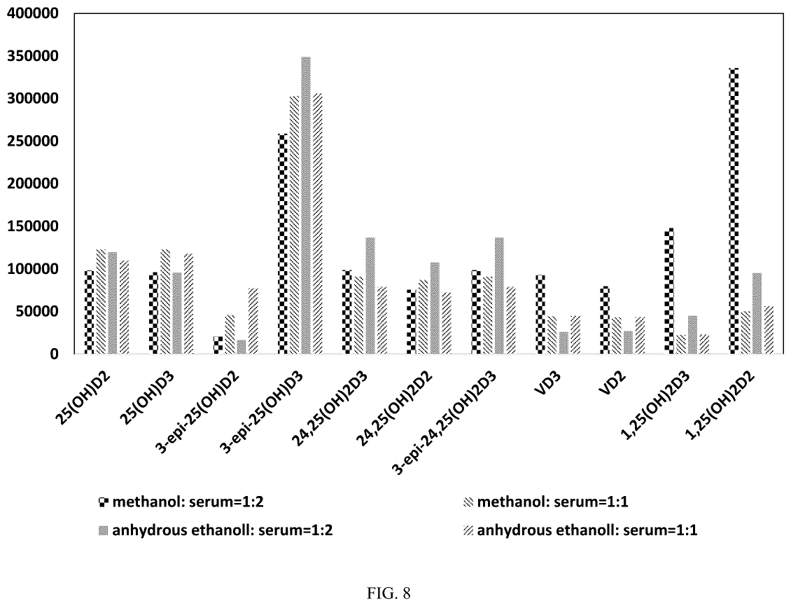

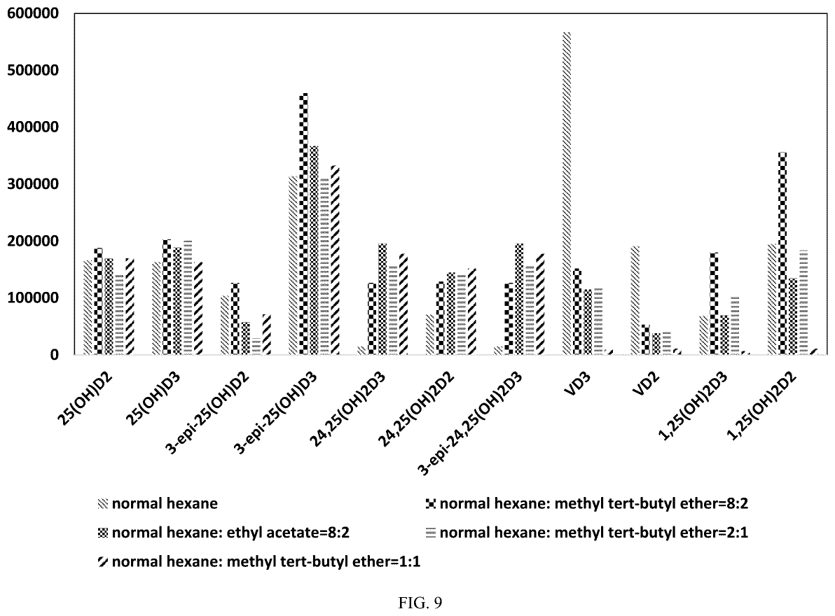

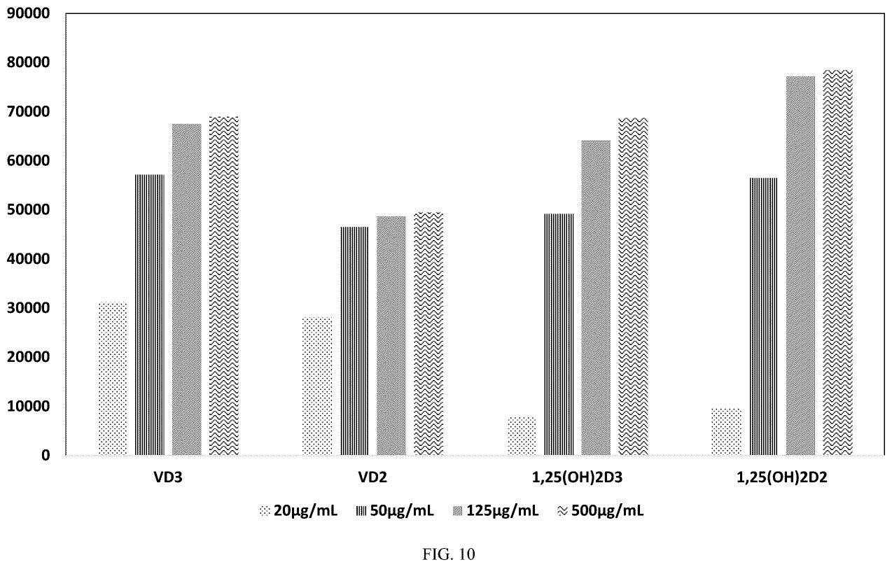

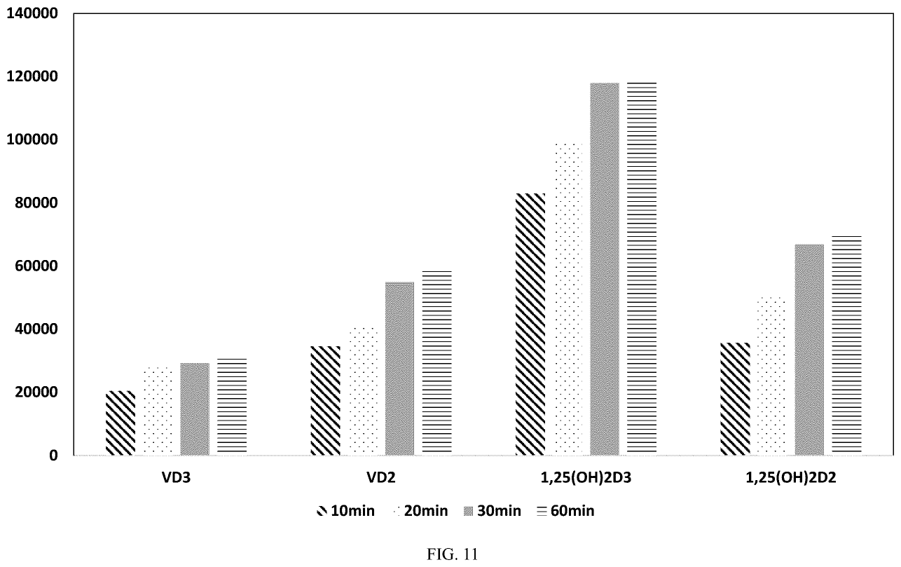

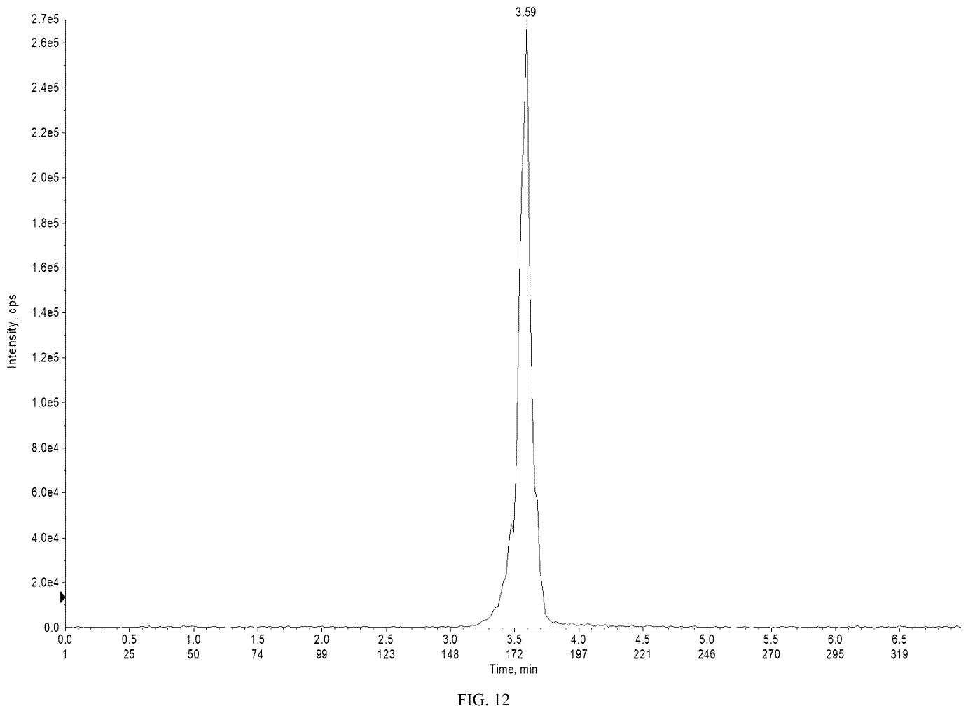

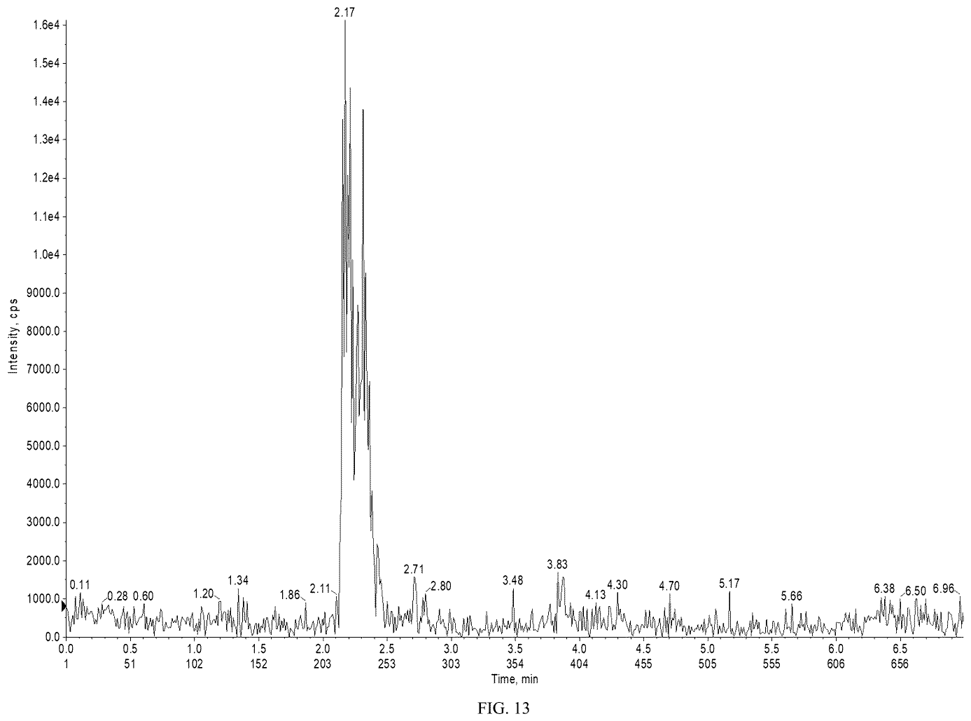

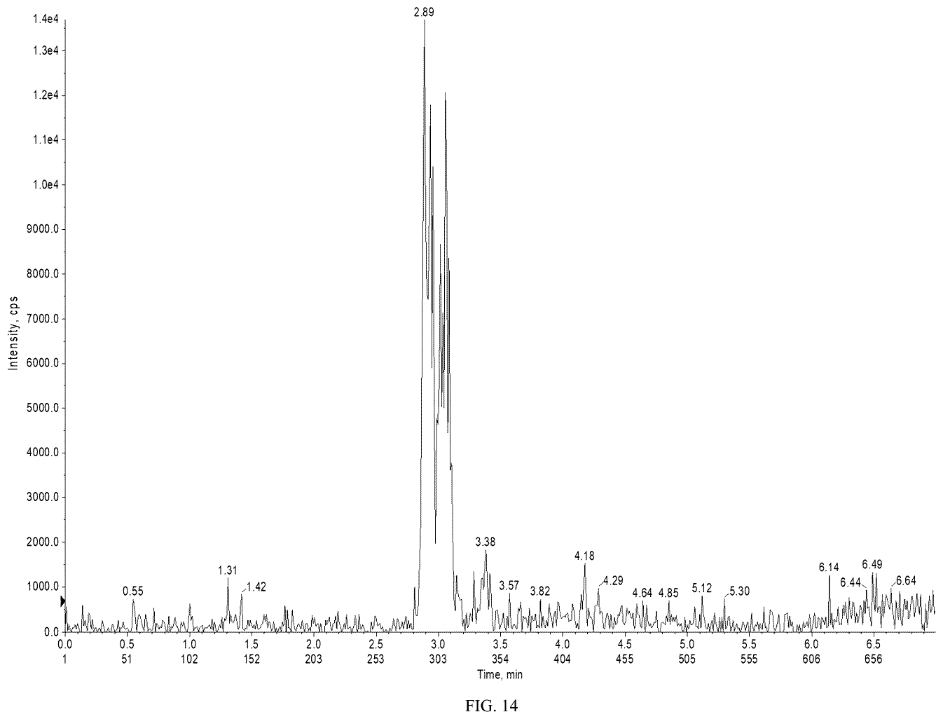

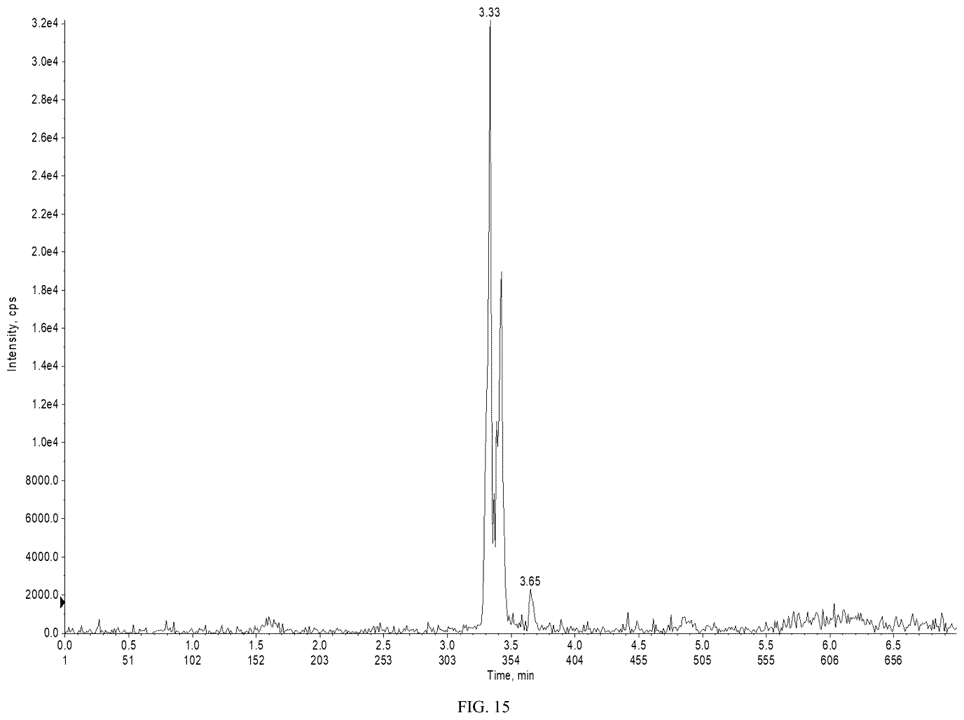

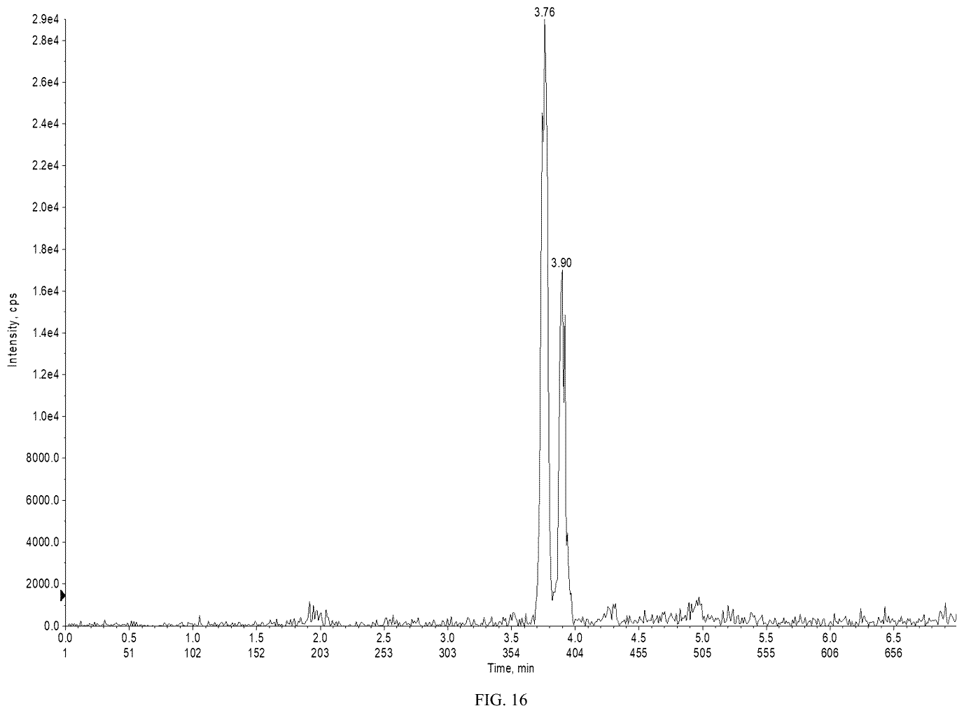

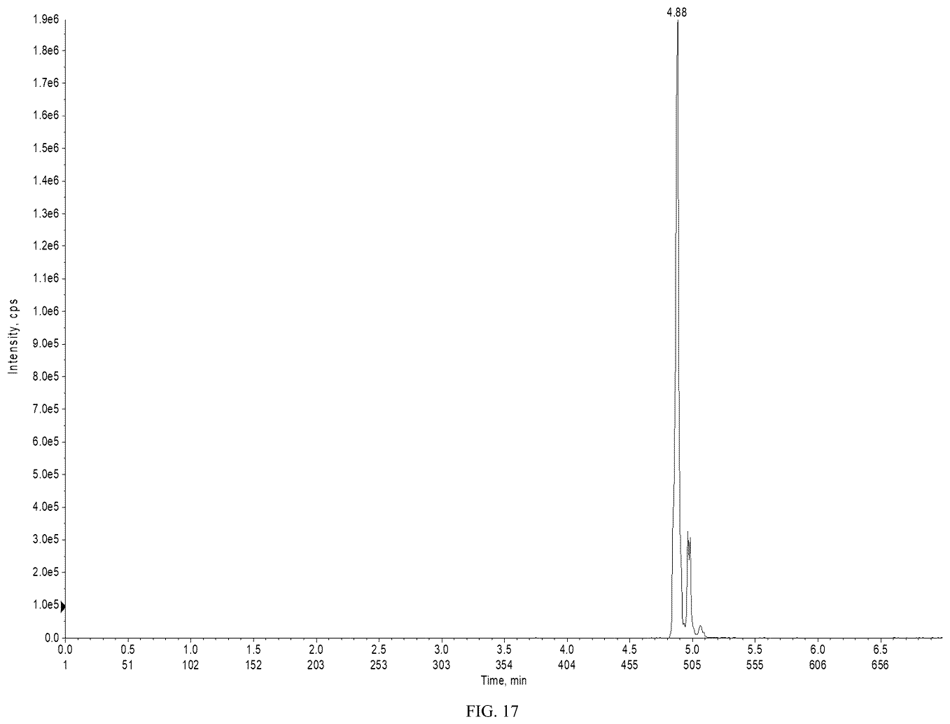

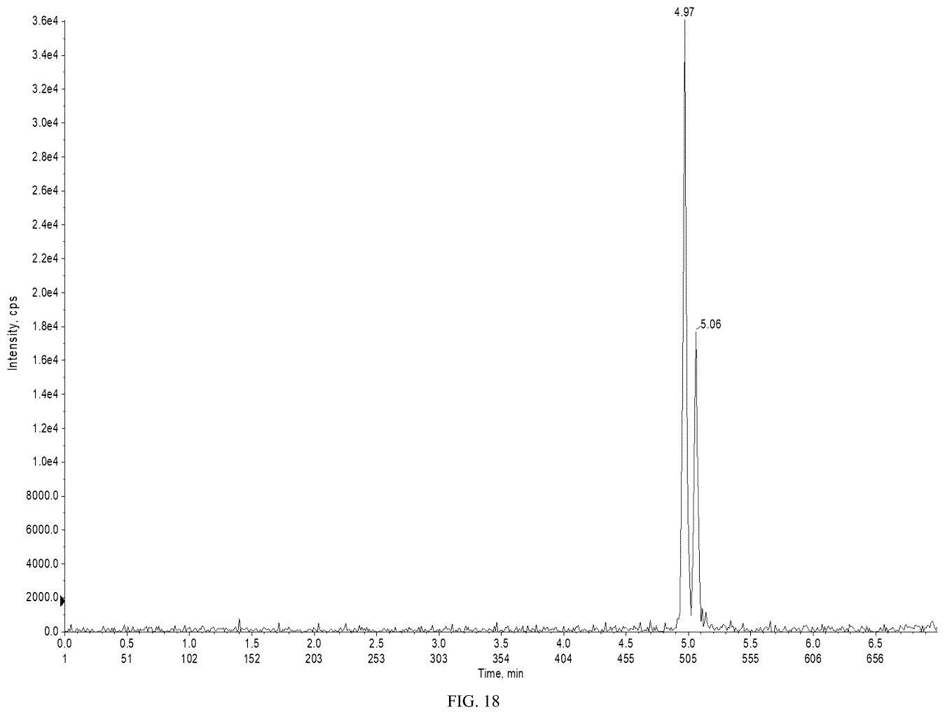

FIGS. 6A-6H were the chromatograms of 11 vitamins D in a serum sample of healthy people. It could be seen from FIGS. 6A-6H that, when the method of the present invention was used in the detection of actual clinical samples, all 11 vitamins D were detected, and all the three groups of epimers were separated by chromatography without impurity interference.

FIGS. 7A-7H were the chromatographic peaks of 11 vitamins D in a standard curve point L5. It could be seen from FIGS. 7A-7H that the chromatographic resolution of each substance to be detected was good.

### Embodiment 2

The technical method in Example 1 was demonstrate as follows:

### I. Linearity of the Method

11 vitamins D were diluted into eight standard curve working liquids with different concentrations in proportion, and detected according to the method of Embodiment 1. With a ratio of a quantitative ion peak area of a target analyte to a peak area of a corresponding isotopic internal standard as an ordinate and a concentration of the standard substance as an abscissa, a standard curve was drawn, and 1/x2 was used for curve weight when fitting the standard curve. An obtained linear range was as shown in Table 13.

TABLE 13

| | | | Linear |
|---|---|---|---|
| Compound | Linear equation | r | range (ng/ml) |
| 25-(OH)D2 | y = 0.464x + 0.00661 | 0.9992 | 0.156~20.000 |
| 25-(OH)D3 | y = 0.767x − 0.0188 | 0.9976 | 1.25~160.00 |
| 3-epi-25-(OH)D2 | y = 0.995x − 0.00309 | 0.9971 | 0.063~8.000 |
| 3-epi-25-(OH)D3 | y = 0.345x + 0.000135 | 0.9976 | 0.25~32.00 |
| 24,25-(OH)₂D2 | y = 2.56x − 0.0363 | 0.9974 | 0.063~8.000 |
| 24,25-(OH)₂D3 | y = 1.02x + 0.00258 | 0.9978 | 0.063~8.000 |
| 3-epi-24,25-(OH)₂D3 | y = 1.39x − 0.00245 | 0.9962 | 0.031~4.000 |
| D2 | y = 0.512x + 0.00581 | 0.9975 | 0.156~20.000 |
| D3 | y = 0.164x + 0.00866 | 0.9981 | 0.313~40.000 |
| 1,25-(OH)₂D2 | y = 2.67x − 0.00711 | 0.9991 | 0.05~6.40 |
| 1,25-(OH)₂D3 | y = 8.8x + 0.0193 | 0.9983 | 0.01~1.28 |

It could be seen from Table 13 that linear relationships of the 11 target analytes were good in the respective linear ranges thereof (r>0.9962).

### II. Recovery Rate and Precision of the Method

11 vitamin D standard working liquids were prepared into three concentrations-; low, medium and high, and recovery rate and precision experiments were carried out. The samples were determined according to the method in Example 1, and five batches were repeatedly analyzed and determined. The recovery rates and precision of 11 vitamins D were shown in Table 14 respectively.

TABLE 14
standard recovery rate and precision of 11 vitamins D

| 25-(OH)D2 | 0.3125 ng/mL | 2.5 ng/mL | 10 ng/mL |
|---|---|---|---|
| Average recovery rate | 105.66 | 102.80 | 101.37 |
| Precision RSD | 5.65% | 5.41% | 1.27% |
| 25-(OH)D3 | 2.5 ng/mL | 20 ng/mL | 80 ng/mL |
| Average recovery rate | 105.16 | 96.01 | 99.26 |
| Precision RSD | 6.10% | 5.32% | 4.30% |
| 3-epi-25-(OH)D2 | 0.125 ng/mL | 1 ng/mL | 4 ng/mL |
| Average recovery rate | 100.00 | 102.00 | 99.56 |
| Precision RSD | 4.62% | 5.49% | 2.79% |
| 3-epi-25-(OH)D3 | 0.5 ng/mL | 4 ng/mL | 16 ng/mL |
| Average recovery rate | 102.08 | 103.94 | 97.84 |
| Precision RSD | 6.15% | 1.58% | 4.67% |
| 24,25-(OH)₂D2 | 0.125 ng/mL | 1 ng/mL | 4 ng/mL |
| Average recovery rate | 95.35 | 104.25 | 101.75 |
| Precision RSD | 3.98% | 3.45% | 1.57% |
| 24,25-(OH)₂D3 | 0.125 ng/mL | 1 ng/mL | 4 ng/mL |
| Average recovery rate | 106.67 | 100.50 | 101.06 |
| Precision RSD | 1.13% | 4.10% | 5.96% |
| 3-epi-24,25-(OH)₂D3 | 0.063 ng/mL | 0.5 ng/mL | 2.0 ng/mL |
| Average recovery rate | 105.56 | 104.60 | 102.81 |
| Precision RSD | 3.26% | 3.53% | 4.83% |
| D3 | 2.5 ng/mL | 20 ng/mL | 80 ng/mL |
| Average recovery rate | 98.80 | 94.23 | 97.89 |
| Precision RSD | 1.43% | 5.94% | 1.15% |
| D2 | 0.313 ng/mL | 2.5 ng/mL | 10 ng/mL |
| Average recovery rate | 95.85 | 103.40 | 98.40 |
| Precision RSD | 5.89% | 3.52% | 2.46% |
| 1,25-(OH)₂D2 | 0.1 ng/mL | 0.8 ng/mL | 3.2 ng/mL |
| Average recovery rate | 100.00 | 106.88 | 101.09 |
| Precision RSD | 5.71% | 5.79% | 1.97% |
| 1,25-(OH)₂D3 | 0.02 ng/mL | 0.16 ng/mL | 0.64 ng/mL |
| Average recovery rate | 103.13 | 103.33 | 95.31 |
| Precision RSD | 4.29% | 4.56% | 4.18% |

Note: The isotope internal standard of 3-epi-24,25-(OH)₂D3 was not purchased, and 24,25-(OH)₂D3-D6 was used as the internal standard thereof in methodological evaluation.

Based on the above verification tests, the average recovery rate of the 11 vitamins D in the range of low, medium and high addition levels was 94.23%-106.88%, and the relative standard deviation was 1.13%-6.15%. The technical indexes such as recovery rate and precision met the requirements. The method for detecting the content of 11 vitamins D in blood had good reproducibility and high recovery rate, which improved the accuracy of the detection results.

### III. Quantitative Limit and Detection Limit of the Method

The detection limit was estimated by the signal-to-noise ratio (3 times S/N) of the low concentration point of the standard curve; the contents of 11 vitamins D in actual serum samples were determined, diluted with 4% BSA in different proportions according to the estimated detection limit concentration, and carry out detection according to the method in Embodiment 1. Each concentration was repeatedly detected for 6 times, it was satisfied that the relative standard deviation was less than or equal to 20%, and the lowest concentration with signal-to-noise ratio greater than 10:1 was set as the quantitative lower limit, and the experimental results obtained were shown in Table 15.

TABLE 15

| | Quantitative | Detection |
|---|---|---|
| Target | limit (ng/mL) | limit (pg/mL) |
| 25-(OH)D2 | 0.10 | 30 |
| 25-(OH)D3 | 0.16 | 50 |
| 3-epi-25-(OH)D2 | 0.05 | 16 |
| 3-epi-25-(OH)D3 | 0.12 | 40 |
| 24,25-(OH)₂D2 | 0.05 | 16 |
| 24,25-(OH)₂D3 | 0.05 | 16 |
| 3-epi-24,25-(OH)₂D3 | 0.03 | 1 |
| D2 | 0.09 | 30 |
| D3 | 0.1 | 30 |
| 1,25-(OH)₂D2 | 0.02 | 7 |
| 1,25-(OH)₂D3 | 0.01 | 3 |

### Embodiment 3

The embodiment was used to illustrate a comparative experiment of the previous treatment method.

The detection was carried out according to the method of Embodiment 1, with a difference that protein precipitants were different in type and dosage. Specifically, methanol and anhydrous ethanol were selected as the protein precipitants respectively, and ratios of addition volumes of the protein precipitants to a serum volume were 1:2 and 1:1 respectively. Internal standard response values of 11 vitamins of the samples after pre-treatment were compared, and a comparison diagram of the experimental results was shown in FIG. 8.

It could be seen from FIG. 8 that, for 25-(OH)D2 and 25-(OH)D3, when the protein precipitant was that methanol:serum=1:1, the response was optimal; for 3-epi-25-(OH)D2, when the protein precipitant was that anhydrous ethanol:serum=1:1, the response was optimal; for 3-epi-25-(OH)D3, 24,25-(OH)₂D3, 24,25-(OH)₂D2 and 3-epi-24,25(OH)₂D3, when the protein precipitant was that anhydrous ethanol:serum=1:2, the response was optimal; and for D3, D2, 1,25-(OH)₂D3 and 1,25-(OH)₂D2, when the protein precipitant was that methanol:serum=1:2, the response was optimal.

In actual blood samples, contents of 1,25-(OH)₂D3 and 1,25-(OH)₂D2 in 11 vitamins D were the lowest, so that a final dosage of the protein precipitant was selected to be that methanol:serum=1:2 under overall consideration.

### Embodiment 4

The embodiment was used to illustrate comparative experiments of extractants and derivation reagents and conditions.

### 1. Comparison of Extraction Effects of Different Extractants:

Three extractants were selected, which were n-hexane, a mixed solvent of n-hexane: methyl tert-butyl ether=4:1, a mixed solvent of n-hexane: ethyl acetate=4:1, a mixed solvent of n-hexane: methyl tert-butyl ether=2:1, and a mixed solvent of n-hexane: methyl tert-butyl ether=1:1. Internal standard response values of 11 vitamins of samples after pre-treatment were compared, and the specific results were shown in FIG. 9.

It can be seen from FIG. 9 that when the extractant was n-hexane, the responses of 24,25-(OH)₂D3 and 3-epi-24,25-(OH)₂D3 were too low to meet quantitative requirements. When the extractant was the mixed solvent of n-hexane: methyl tert-butyl ether=l: 1, the responses of D3, D2, 1,25-(OH)₂D3 and 1,25-(OH)₂D2 were too low to meet the quantitative requirements. When the extractant was the mixed solvent of n-hexane: ethyl acetate=4:1, the responses of 3-epi-25-(OH)D3, 1,25-(OH)₂D3 and 1,25-(OH)₂D2 were too low to meet the quantitative requirements. When the extractant was the mixed solvent of n-hexane: methyl tert-butyl ether=2: I to 4:1, the responses and sensitivities of 11 vitamins could be taken into account, and based on the fact that, in actual blood samples, contents of 1,25-(OH)₂D3 and 1,25-(OH)₂D2 in 11 vitamins D were the lowest, the final extractant was n-hexane: methyl tert-butyl ether=4:1 under overall consideration.

### 2. Comparison of Derivation Reagent Concentrations:

PATD was selected as the extractant to carry out an optimal experiment on dosages of the derivation reagent. The dosages of the derivation reagent were 20 μg/mL, 50 μg/mL, 125 μg/mL and 500 μg/mL respectively, and the specific optimization results were shown in FIG. 10.

It could be seen from FIG. 10 that, with the increase of concentration of the derivation reagent, the responses of four substances were continuously increased obviously and tended to be stable, and the concentration of the derivation reagent was determined to be 50 μg/mL to 500 μg/mL.

In order to give consideration to the overall response of four substances and consider the cost, the concentration of the derivation reagent was finally selected to be 125 μg/mL.

### 3. Comparison of Derivation Time:

The derivation times of 10 min, 20 min, 30 min and 60 min were investigated respectively, and the specific optimization results of the derivation times were shown in FIG. 11.

It could be seen from FIG. 11 that, with the extension of the derivation time, the responses of four vitamins were gradually increased and tended to be stable, and the derivation time was selected to be 20 min to 60 min. When the derivation time was 30 min, the responses of four vitamins were not increased obviously even if the derivation time was extended. Considering the sensitivity and pre-treatment time comprehensively, the derivation time was finally selected to be 30 min.

### Embodiment 5

The embodiment was used to illustrate comparative experiments of liquid phase conditions.
D1. The method of Embodiment 1 was used to detect an L8 point of a standard curve, with a difference that a gradient elution method shown in Table 16 was used.

TABLE 16

| Time/ | Phase | Phase |
|---|---|---|
| min | A | B |
| 0.00 | 33 | 77 |
| 2.00 | 33 | 77 |
| 2.01 | 0 | 100 |
| 3.25 | 0 | 100 |
| 3.26 | 33 | 77 |
| 7.00 | 33 | 77 |

The obtained chromatogram was shown in FIG. 12.

It could be seen from FIG. 12 that 25-(OH)D3, 3-epi-25-(OH)D3, 25-(OH)D2, 3-epi-25-(OH)D2, 24,25-(OH)₂D3 and 3-epi-24,25(OH)₂D3 were all inseparable under the conditions.
D2. The method of Embodiment 1 was used to detect an L8 point of a standard curve, with a difference that a gradient elution method shown in Table 17 was used.

TABLE 17

| Time/ | Phase | Phase |
|---|---|---|
| min | A | B |
| 0.00 | 25 | 75 |
| 1.00 | 25 | 75 |
| 2.50 | 15 | 85 |
| 4.50 | 15 | 85 |
| 4.60 | 0 | 100 |
| 5.50 | 0 | 100 |
| 5.51 | 25 | 75 |
| 7.00 | 25 | 75 |

The obtained chromatogram was shown in FIG. 13.

It could be seen from FIG. 13 that 24,25-(OH)₂D3 and 3-epi-24,25(OH)₂D3 were inseparable under the conditions.
D3. The method of Embodiment 1 was used to detect an L8 point of a standard curve, with a difference that a gradient elution method shown in Table 18 was used.

TABLE 18

| Time/ | Phase | Phase |
|---|---|---|
| min | A | B |
| 0.00 | 28 | 72 |
| 1.00 | 28 | 12 |
| 2.50 | 15 | 85 |
| 4.50 | 15 | 85 |
| 4.60 | 0 | 100 |
| 5.50 | 0 | 100 |
| 5.51 | 28 | 72 |
| 7.00 | 28 | 72 |

The obtained chromatogram was shown in FIG. 14.

It could be seen from FIG. 14 that 24,25-(OH)₂D3 and 3-epi-24,25(OH)₂D3 were inseparable under the conditions.
D4. The method of Embodiment 1 was used to detect an L8 point of a standard curve, with a difference that a gradient elution method shown in Table 19 was used.

TABLE 19

| Time/ | Phase | Phase |
|---|---|---|
| min | A | B |
| 0.00 | 30 | 70 |
| 1.00 | 30 | 70 |
| 2.50 | 15 | 85 |
| 4.50 | 15 | 85 |
| 4.60 | 0 | 100 |
| 5.50 | 0 | 100 |
| 5.51 | 30 | 70 |
| 7.00 | 30 | 70 |

The obtained chromatogram was shown in FIG. 15.

It could be seen from FIG. 15 24,25-(OH)₂D3 and 3-epi-24,25(OH)₂D3 were basically separable under the conditions.
D5. The method of Embodiment 1 was used to detect an L8 point of a standard curve, with a difference that a gradient elution method shown in Table 20 was used.

TABLE 20

| Time/ | Phase | Phase |
|---|---|---|
| min | A | B |
| 0.50 | 35 | 65 |
| 1.50 | 25 | 75 |
| 2.50 | 25 | 75 |
| 3.00 | 15 | 85 |
| 5.00 | 15 | 85 |
| 5.10 | 0 | 100 |
| 6.00 | 0 | 100 |
| 6.10 | 35 | 65 |
| 7.50 | 35 | 65 |

The obtained chromatograms were shown in FIG. 16 to FIG. 18.

It could be seen from FIG. 16 to FIG. 18 that baseline separation of 25-(OH)D3, 3-epi-25-(OH)D3, 25-(OH)D2, 3-epi-25-(OH)D2, 24,25-(OH)₂D3 and 3-epi-24,25-(OH)₂D3 were realized under the elution conditions.

### Embodiment 6

A pure product, a sample and an L8 point of a standard curve were detected according to the method of Embodiment 1, while the differences were that: (2) the calibration of the standard curve working liquid and (3) the treatment of sample to be detected were carried out by the following methods.

Compared with the calibration method of the standard curve working liquid: 20 μL of standard working liquid I, 10 μL of internal standard working liquid I, 20 μL of standard working liquid II and 10 μL of internal standard working liquid II were respectively taken by a pipette to prepare eight standard curve working liquids with different concentrations, blow-dried by nitrogen, added with 70 μL of 70% methanol aqueous solution (0.1% formic acid), and subjected to vortex mixing at a rotating speed of 2,000 rpm for 1 min.

Compared with the pre-treatment conditions: 10 μL of internal standard working liquid I and 10 μL of internal standard working liquid II were respectively taken with a pipette first and then put into a 1.5 mL centrifuge tube, 200 μL of serum sample to be detected was taken by a pipette, added with 100 μL of methanol, subjected to vortex mixing at a rotating speed of 2,000 rpm for 3 min, added with 650 μL of n-hexane: methyl tert-butyl ether=4:1, subjected to vortex mixing at a rotating speed of 2,000 rpm for 5 min, and centrifuged at a rotating speed of 14,000 rpm for 10 min, 600 μL of supernatant was added into a clean 1.5 mL centrifuge tube, and then 650 μL of n-hexane: methyl tert-butyl ether=4:1 was added into the 1.5 mL centrifuge tube, subjected to vortex mixing at a rotating speed of 2,000 rpm for 5 min, and centrifuged at a rotating speed of 14,000 rpm for 10 min to obtain a supernatant. The supernatant was added with 100 μL of 70% methanol aqueous solution (containing 0.1% formic acid) for redissolution, subjected to vortex mixing at a rotating speed of 2,000 rpm for 1 min, centrifuged at a rotating speed of 14,000 rpm for 5 min, and 90 μL of supernatant was taken as a solution to be detected.

Response values of D2, D3, 1,25-(OH)₂D2 and 1,25-(OH)₂D3 were measured. Meanwhile, the same samples were detected according to the method of Embodiment 1 to measure the response values. Obtained experimental results were as shown in Table 21 and Table 22.

TABLE 21

| No derivative treatment | | After derivative treatment | |
|---|---|---|---|
| Response of D3 internal | 2.97 × 10⁶ | Response of D3 | 2.99 × 10⁷ |
| standard in pure product | | internal standard in | |
| | | pure product | |
| Response of D2 internal | 3.01 × 10⁵ | Response of D2 | 2.87 × 10⁶ |
| standard in pure product | | internal standard in | |
| | | pure product | |
| Response of D3 internal | 1.20 × 10⁴ | Response of D3 | 6.55 × 10⁶ |
| standard in sample | | internal standard in | |
| | | sample | |
| Response of D2 internal | 2.45 × 10³ | Response of D2 | 1.01 × 10⁶ |
| standard in sample | | internal standard in | |
| | | sample | |

TABLE 22

| No derivative treatment | | After derivative treatment | |
|---|---|---|---|
| Response of 1,25- | 1.5 × 10⁴ | Response of 1,25- | 1.5 × 10⁶ |
| (OH)₂D3 of L8 point | | (OH)₂D3 of L8 point | |
| of standard curve | | of standard curve | |
| Response of 1,25- | 4.4 × 10⁵ | Response of 1,25- | 2.1 × 10⁶ |
| (OH)₂D2 of L8 point | | (OH)₂D2 of L8 point | |
| of standard curve | | of standard curve | |

It could be seen from Table 21 and Table 22 that the response values of the four substances of D2, D3, 1,25-(OH)₂D2 and 1,25-(OH)₂D3 after the derivatization treatment were significantly increased, and matrix inhibition in D2 and D3 samples was significantly improved.

The above are only specific embodiments of the present invention, so that those skilled in the art can understand or realize the present invention. Various modifications to these embodiments will be apparent to those skilled in the art, and the generic principles defined herein may be embodied in other embodiments without departing from the spirit or scope of the present invention. Therefore, the present invention will not to be limited to these embodiments shown herein, but is to be in conformity with the widest scope consistent with the principles and novel features disclosed herein.

What is claimed is:

1. A method for simultaneously detecting 11 vitamins D by a Liquid Chromatography Tandem-mass Spectrometry (LC-MS/MS), wherein the 11 vitamins D comprises D2, D3, 1,25-(OH)₂D2, 1,25-(OH)₂D3, 25-(OH)D2, 25-(OH)D3, 3-epi-25-(OH)D2, 3-epi-25-(OH)D3, 24,25-(OH)₂D2, 24,25-(OH)₂D3, and 3-epi-24,25-(OH)₂D3, and the method at least comprises the following steps of:
S1. preparing a standard curve equation, comprising:
preparing an internal standard working liquid and a standard working liquid, preparing a sample solution for standard curve, and detecting the sample solution for standard curve by using the LC-MS/MS to obtain the standard curve equation for calculating contents of the 11 vitamins D in blood;
S2. pre-treating a sample to be detected, comprising:
uniformly mixing the internal standard working liquid, the sample to be detected, and a protein precipitant to obtain a first resulting mixture, extracting the first resulting mixture twice with an extractant to obtain a total supernatant, adding a derivatization reagent to a part of the total supernatant to obtain a second resulting mixture, uniformly mixing the second resulting mixture for a derivatization treatment to obtain a third resulting mixture, blow-drying the third resulting mixture, and adding a remaining part of the total supernatant to the third resulting mixture to obtain a fourth resulting mixture, blow-drying the fourth resulting mixture again, adding a reconstitution solution to the fourth resulting mixture to obtain a fifth resulting mixture, uniformly mixing the fifth resulting mixture, and centrifuging the fifth resulting mixture and taking a supernatant of the fifth resulting mixture as an incoming sample;
the extractant is a mixed solution of normal hexane and methyl tert-butyl ether; and
S3. detecting the incoming sample, comprising:
detecting the incoming sample by using the LC-MS/MS, and substituting a detection result of the incoming sample into the standard curve equation to obtain the contents of the 11 vitamins D in the sample to be detected.

2. The method according to claim 1, wherein SI comprises:
S11. respectively preparing a first internal standard working liquid, a second internal standard working liquid, a first standard working liquid, and a second standard working liquid;
the first internal standard working liquid containing isotopic internal standards of D2, D3, 1,25-(OH)₂D2, and 1,25-(OH)₂D3; the second internal standard working liquid containing isotopic internal standards of 25-(OH)D2, 25-(OH)D3, 3-epi-25-(OH)D2, 3-epi-25-(OH)D3, 24,25-(OH)₂D2, and 24,25-(OH)₂D3;
the first standard working liquid containing standard solutions of D2, D3, 1,25-(OH)₂D2, and 1,25(OH)₂D3; and
the second standard working liquid containing standard solutions of 25-(OH)D2, 25-(OH)D3, 3-epi-25-(OH)D2, 3-epi-25-(OH)D3, 24,25-(OH)₂D2, 24,25-(OH)₂D3, and 3-epi-24,25-(OH)₂D3;
S12. mixing the first standard working liquid and the first internal standard working liquid to prepare a standard curve working liquid with a gradient concentration, blow-drying the standard curve working liquid, adding the derivatization reagent to the standard curve working liquid for the derivatization treatment to obtain a derived standard curve working liquid, blow-drying the derived standard curve working liquid, respectively adding the second standard working liquid and the second internal standard working liquid with corresponding concentrations according to a concentration gradient to obtain a sixth resulting mixture, adding the reconstitution solution to the sixth resulting mixture to obtain a seventh resulting mixture, and mixing the seventh resulting mixture to obtain the sample solution for standard curve; and
S13. detecting the sample solution for standard curve by using the LC-MS/MS to obtain the standard curve equation.

3. The method according to claim 2, wherein:
a preparation method of the first standard working liquid is: dissolving D2 and D3 solid standards with anhydrous ethanol or methanol to obtain a first mother liquid, mixing the first mother liquid with 1,25-(OH)₂D2 and 1,25-(OH)₂D3 liquid standards with known concentrations to obtain an eighth resulting mixture, and continuously diluting the eighth resulting mixture with the anhydrous ethanol or the methanol to obtain the first standard working liquid;
a preparation method of the second standard working liquid is: dissolving 24,25-(OH)₂D2 solid standard with a 70%-100% methanol aqueous solution or a 70%-100% ethanol aqueous solution by volume to obtain a second mother liquid, and mixing the second mother liquid with 25-(OH)D2, 25-(OH)D3, 3-epi-25-(OH)D2, 3-epi-25-(OH)D3, 24,25-(OH)₂D3 and 3-epi-24,25-(OH)₂D3 with known concentrations to obtain a ninth resulting mixture, and continuously diluting the ninth resulting mixture with the 70%-100% methanol aqueous solution or the 70%-100% ethanol aqueous solution by volume to obtain the second standard working liquid;
a preparation method of the first internal standard working liquid is: diluting commercially available liquid standards of isotopic internal standards of D2, D3, 1,25-(OH)₂D2, and 1,25-(OH)₂D3 with the anhydrous ethanol or the methanol to obtain the first internal standard working liquid; and
a preparation method of the second internal standard working liquid is: dissolving solid standards of isotopic internal standards of 25-(OH)D3, 24,25-(OH)₂D3, and 24,25-(OH)₂D2 with the 70′%-100% methanol aqueous solution or the 70%-100% ethanol aqueous solution by volume to obtain a third mother liquid, and mixing the third mother liquid with liquid standards of isotopic internal standards of 25-(OH)D2, 3-epi-25-(OH)D2, 3-epi-25-(OH)D3, and 3-epi-24,25-(OH)₂D3 with known concentrations to obtain a tenth resulting mixture, and continuously diluting the tenth resulting mixture with the 70%/0-100%/o methanol aqueous solution or the 70%-100% ethanol aqueous solution by volume to obtain the second internal standard working liquid.

4. The method according to claim 1, wherein:
the derivatization reagent is selected from a 50-500 μg/mL 4-Phenyl-3H-1,2,4-triazole-3,5(4H)-dione (PTAD) solution, and the derivatization treatment lasts for 20-60 min;
the reconstitution solution is selected from an aqueous solution containing 0.05%-0.2% formic acid by volume and 60%-100% methanol by volume;
the protein precipitant is selected from methanol and anhydrous ethanol; and
the extractant is the mixed solvent of normal hexane and methyl tert-butyl ether with a volume ratio of 2:1-4:1.

5. The method according to claim 2, wherein in S12:
a volume ratio of the first standard working liquid to the first internal standard working liquid is 1:1-2:1;
a volume ratio of the first internal standard working liquid to the derivatization reagent is 1:10-1:20;
a volume ratio of the first standard working liquid to the second standard working liquid is 1:1; and
a volume ratio of the second standard working liquid to the second internal standard working liquid II and the reconstitution solution is 2:1:7.

6. The method according to claim 5, wherein in S12, the derivatization reagent is added, and after a vortex mixing at a rotating speed of 1,500-2,500 rpm for 30 s to 1 min, the derivatization treatment is carried out, and after adding the reconstitution solution, the vortex mixing is carried out at a rotating speed of 1,500-2,500 rpm for 1-3 min to obtain the sample solution for standard curve.

7. The method according to claim 2, wherein in S2:
the first internal standard working liquid, the second internal standard working liquid, the sample to be detected, and the protein precipitant are mixed, and a volume ratio of the first internal standard working liquid to the second internal standard working liquid is 1:1;
a volume ratio of the sample to be detected to the protein precipitant is 1:1-2:1;
a volume ratio of the part of the total supernatant for the derivatization treatment to the remaining part of the total supernatant is 1:1; and
a volume ratio of the reconstitution solution to the sample to be detected is 1:1-1:2.

8. The method according to claim 7, wherein in S2:
carrying out a vortex mixing the first internal standard working liquid, the second internal standard working liquid, the sample to be detected and the protein precipitant at a rotating speed of 1,500-2,500 rpm for 3-5 min to obtain the first resulting mixture, extracting the first resulting mixture twice with the extractant to obtain the total supernatant, adding the derivatization reagent to the part of the total supernatant to obtain the second resulting mixture, carrying out the derivatization treatment on the second resulting mixture after the vortex mixing at the rotating speed of 1,500-2,500 rpm for 30 s to 1 min to obtain the third resulting mixture, blow-drying the third resulting mixture, adding the remaining part of the total supernatant to the third resulting mixture to obtain the fourth resulting mixture, blow-drying again the fourth resulting mixture, adding the reconstitution solution to the fourth resulting mixture to obtain the fifth resulting mixture, carrying out the vortex mixing at the rotating speed of 1,500-2,500 rpm for 1-3 min on the fifth resulting mixture, centrifuging the fifth resulting mixture at a rotating speed of 12,000-14,000 rpm for 5-10 min, and taking the supernatant of the fifth resulting mixture as the incoming sample.

9. The method according to claim 1, wherein in S2, extracting twice with the extractant comprises:
during a first extraction, a volume ratio of the sample to be detected to the extractant is 2:6.5-1:5, and
a volume ratio of the extractant used during a second extraction to the extractant used during the first extraction is 0.8-1:1.

10. The method according to claim 9, wherein during the first extraction, the internal standard working liquid, the sample to be detected, and the protein precipitant are mixed uniformly to obtain the first resulting mixture, and then the extractant is added to the first resulting mixture to obtain a sixth resulting mixture, and the sixth resulting mixture is subjected to a vortex mixing at a rotating speed of 1,500-25,00 rpm for 3-5 min, and centrifuged at a rotating speed of 12,000-14,000 rpm for 5-10 min, and a supernatant of the sixth resulting mixture is taken as a first supernatant; the extractant is added into a centrifuged precipitate of the sixth resulting mixture to obtain a seventh resulting mixture, subjected to the vortex mixing at the rotating speed of 1,500-2,500 rpm for 3-5 min, centrifuged at the rotating speed of 12,000-14,000 rpm for 5-10 min, a supernatant of the seventh resulting mixture is taken as a second supernatant, and the first supernatant and the second supernatant are combined to obtain the total supernatant.

11. The method according to claim 1, wherein the LC-MS/MS is used for a detection under the following high-performance liquid phase conditions:
a pentafluorophenyl chromatographic column is adopted as a chromatographic column;
mobile phases: a phase A is an aqueous solution containing 0.05%-0.2% formic acid and 1 mM-5 mM ammonium formate or ammonium acetate; and a phase B is a methanol solution containing 0.05%-0.2% formic acid and 1 mM-5 mM ammonium formate or ammonium acetate;
a flow rate: 0.25-0.35 mL/min, a column temperature: 25-35° C., a sample volume: 10-20 μL, and an analysis time: 8 min; and
gradient elution conditions are as follows:
at 0-1.50 min, the phase A changes from a concentration A1 to a concentration A2 at a constant speed, and the phase B changes from a concentration B1 to a concentration B2 at a constant speed;
at 1.50-2.50 min, the phase A adopts the concentration A2, and the phase B adopts the concentration B2;
at 2.50-3.00 min, the phase A changes from the concentration A2 to a concentration A3 at a constant speed, and the phase B changes from the concentration B2 to a concentration B3 at a constant speed;
at 3.00-5.00 min, the phase A adopts the concentration A3, and the phase B adopts the concentration B3;
at 5.00-5.10 min, the phase A changes from the concentration A3 to a concentration A4 at a constant speed, and the phase B changes from the concentration B3 to a concentration B4 at a constant speed;
at 5.10-6.50 min, the phase A adopts the concentration A4, and the phase B adopts the concentration B4;
at 6.51-8.00 min, the phase A adopts a concentration A5, and the phase B adopts a concentration B5;
the concentration A1 is selected from 40%-30%, the concentration B1 is selected from 60%-70/6, and the concentration A1+the concentration B1=100%;
the concentration A2 is selected from 28%-24%, the concentration B2 is selected from 72%-76%, and the concentration A2+the concentration B2=100%;
the concentration A3 is selected from 22%-15%, the concentration B3 is selected from 78%-85%, and the concentration A3+the concentration B3=100%;
the concentration A4 is selected from 10/6-0, the concentration B4 is selected from 90%-100%, and the concentration A4+the concentration B4=100%; and
the concentration A5 is selected from 40/6-30%, the concentration B5 is selected from 60%-70%, and the concentration A5+the concentration B5=100%.

12. The method according to claim 1, wherein the LC-MS/MS is used for a detection under the following mass spectrum conditions:
using an electrospray ion source (ESI) and a positive ion mode for a multi-reaction monitoring under an ionspray voltage: 5,000 V-5,500 V; an ion source temperature: 300-400° C.; an atomizing gas: 45-55 psi; an auxiliary gas: 25-35 psi; a curtain gas: 20-25 psi; and a collision gas: 8-10 psi;
ion pairs expressed by precursor ion/product ion are:
D2: quantitative ion pair 572.3/298.1, and qualitative ion pair 572.3/280.3;
D3: quantitative ion pair 560.3/298.1, and qualitative ion pair 560.3/365.2;
1,25-(OH)₂D2: quantitative ion pair 586.1/314.3, and qualitative ion pair 604.1/314.3;
1,25-(OH)₂D3: quantitative ion pair 574.3/314.3, and qualitative ion pair 574.3/243.8;
25-(OH)D2: quantitative ion pair 413.2/337.2, and qualitative ion pair 413.2/355.3;
25-(OH)D3: quantitative ion pair 401.3/365.1, and qualitative ion pair 401.3/383.3;
3-epi-25-(OH)D2: quantitative ion pair 413.2/337.2, and qualitative ion pair 413.2/355.3;
3-epi-25-(OH)D3: quantitative ion pair 401.3/365.1, and qualitative ion pair 401.3/355.3;
24,25-(OH)₂D2: quantitative ion pair 393.4/243.4, and qualitative ion pair 393.4/268.1;
24,25-(OH)₂D3: quantitative ion pair 417.2/381.3, and qualitative ion pair 417.2/399.1; and
3-epi-24,25-(OH)₂D3: quantitative ion pair 417.2/381.3, and qualitative ion pair 417.2/399.1.

13. The method according to claim 2, wherein:
the derivatization reagent is selected from a 50-500 μg/mL PTAD solution, and the derivatization treatment lasts for 20-60 min;
the reconstitution solution is selected from an aqueous solution containing 0.05%-0.2% formic acid by volume and 60%-100% methanol by volume;
the protein precipitant is selected from methanol and anhydrous ethanol; and
the extractant is the mixed solvent of normal hexane and methyl tert-butyl ether with a volume ratio of 2:1-4:1.

14. The method according to claim 3, wherein in S12:
a volume ratio of the first standard working liquid to the first internal standard working liquid is 1:1-2:1;
a volume ratio of the first internal standard working liquid to the derivatization reagent is 1:10-1:20;
a volume ratio of the first standard working liquid to the second standard working liquid is 1:1; and
a volume ratio of the second standard working liquid to the second internal standard working liquid II and the reconstitution solution is 2:1:7.

15. The method according to claim 3, wherein in S12:
the first internal standard working liquid, the second internal standard working liquid, the sample to be detected, and the protein precipitant are mixed, and a volume ratio of the first internal standard working liquid to the second internal standard working liquid is 1:1;
a volume ratio of the sample to be detected to the protein precipitant is 1:1-2:1;
a volume ratio of the part of the total supernatant for the derivatization treatment to the remaining part of the total supernatant is 1:1; and
a volume ratio of the reconstitution solution to the sample to be detected is 1:1-1:2.

16. The method according to claim 8, wherein in S2, extracting twice with the extractant comprises:
during a first extraction, a volume ratio of the sample to be detected to the extractant is 2:6.5-1:5; and
a volume ratio of the extractant used during a second extraction to the extractant used during the first extraction is 0.8-1:1.
